(19) United States
(12) Patent Application Publication
(10) Pub. No.: US 2023/0416359 A1
(43) Pub. Date: Dec. 28, 2023

(54) OLFACTORY RECEPTORS FOR USE AS TARGETS FOR ANTIGEN BINDING MOLECULES TO DETECT AND TREAT CANCER

(71) Applicant: H. LEE MOFFITT CANCER CENTER AND RESEARCH INSTITUTE, INC., Tampa, FL (US)

(72) Inventors: Jose CONEJO-GARCIA, Tampa, FL (US); Alexandra MARTIN, Tampa, FL (US)

(21) Appl. No.: 18/011,338

(22) PCT Filed: Jun. 17, 2021
(86) PCT No.: PCT/US2021/037935

§ 371 Date: Dec. 19, 2022

Related U.S. Application Data
(60) Provisional application No. 63/040,082, filed on Jun. 17, 2020.
(60) Provisional application No. 63/082,585, filed on Sep. 24, 2020.
(60) Provisional application No. 63/181,465, filed on Apr. 29, 2021.

(51) Int. Cl. C07K 16/28; G01N 33/574; A61K 39/00; A61P 35/00

(52) U.S. Cl. CPC C07K 16/28; G01N 33/57492; A61K 39/4611; A61K 39/4631; A61K 39/464402; A61P 35/00; C07K 2317/622

(57) ABSTRACT

Disclosed are compositions that selectively bind OR2H1, OR52R1, OR56A3, OR12D2, OR2G3, OR8B2, OR8B3, OR51F1, OR8K3, OR8B4, OR6Q1, OR5M8, and/or OR5V1 and the use of said compositions for the treatment of a cancer. Also disclosed herein are methods for detecting a cancer based on the presence or overexpression of OR2H1, OR52R1, OR56A3, OR12D2, OR2G3, OR8B2, OR8B3, OR51F1, OR8K3, OR8B4, OR6Q1, OR5M8, and/or OR5V1.

This application claims the benefit of U.S. Provisional Application No. 63/181,465, filed on Apr. 29, 2021; U.S. Provisional Application No. 63/082,585, filed on Sep. 24, 2020; and U.S. Provisional Application No. 63/040,082, filed on Jun. 17, 2020, applications which are incorporated herein by reference in their entirety.

This invention was made with Government support under Grant No. CA232758 awarded by the National Institute of Health. The Government has certain rights in the invention.

### I. BACKGROUND

Despite the years and billions of dollars spent trying to devise new ways to treat and prevent cancer, cancer remains the primary health risk world-wide. What are needed are new ways to detect and treat cancer.

### II. REFERENCE TO SEQUENCE LISTING

The sequence listing submitted on Aug. 22, 2023, as an .TXT file entitled “10110_257WO1_ST25.TXT,” created on Jul. 31, 2023, and having a file size of 57,344 bytes is hereby incorporated by reference pursuant to 37 C.F.R. § 1.52(e)(5).

### III. SUMMARY

Disclosed are olfactory receptor binding molecules and methods of their use of the detection and treatment of cancer.

In one aspect, disclosed herein are antigen binding molecules (including, but not limited to RNAi, peptide, protein, chimeric antigen receptor (CAR) T cell, CAR NK cell, CAR macrophage (CARMA), siRNA, immunotoxin, diabody, antibody (including, but not limited to humanized and human IgA, IgG (such as, for example IgG1, GgG2, GgG3, and IgG4), IgE, IgM antibodies), or a functional antibody fragment (including, but not limited to a scFv)) that selective binds to an olfactory receptor selected from the group consisting of OR2H1 (for example a OR2H1 as set forth in SEQ ID NO: 1), OR52R1, OR56A3, OR12D2, OR2G3, OR8B2, OR8B3, OR51F1, OR8K3, OR8B4, OR6Q1, OR5M8, and OR5V1 (for example, an OR5V1 as set forth in SEQ ID NO: 5). For example, disclosed herein are antigen binding molecules that bind to selectively binds to an olfactory receptor OR5V1 comprising an extracellular domain as set forth in SEQ ID NO: 5. In one aspect the antigen binding molecule can comprise one or more complementarity determining regions (CDRs) as set forth in SEQ ID NO: 9, SEQ ID NO: 10, SEQ ID NO: 11, SEQ ID NO: 14, SEQ ID NO: 15, SEQ ID NO: 16, SEQ ID NO: 21, SEQ ID NO: 22, SEQ ID NO: 23, SEQ ID NO: 26, SEQ ID NO: 27, SEQ ID NO: 28, SEQ ID NO: 31, SEQ ID NO: 32, SEQ ID NO: 33, SEQ ID NO: 35, SEQ ID NO: 36, SEQ ID NO: 37, SEQ ID NO: 41, SEQ ID NO: 42, SEQ ID NO: 43, SEQ ID NO: 45, SEQ ID NO: 46, SEQ ID NO: 47, SEQ ID NO: 53, SEQ ID NO: 54, SEQ ID NO: 55, SEQ ID NO: 61, SEQ ID NO: 62, and/or SEQ ID NO: 63.

Also disclosed herein are methods of detecting a cancer in a subject comprising obtaining a tissue sample of a suspected cancerous tissue from the subject and contacting the tissue sample with the antigen binding molecule of any preceding aspect. For example, disclosed herein, in one aspect, are methods of detecting a cancer in a subject comprising obtaining a tissue sample of a suspected cancerous tissue from the subject and assaying for the presence or overexpression of an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and OR5V1; wherein the presence of the olfactory receptor in the tissue sample not present in a negative control tissue sample or overexpression of the olfactory receptor in the tissue sample relative to a negative control tissue sample indicates the presence of a cancer in the subject. In one aspect, the olfactory receptor comprises OR2H1 and the tissue sample is not a testis tissue sample; the olfactory receptor comprises OR52R1; the olfactory receptor comprises OR56A3 and the tissue sample is not a urinary bladder, testis, cervix or placenta tissue sample; the olfactory receptor comprises OR12D2 and the tissue sample is not a testis, fallopian tube, or breast tissue sample; the olfactory receptor comprises OR2G3 and the tissue sample is not a testis, T cell, or urinary bladder tissue sample; the olfactory receptor comprises OR8B3 and the tissue sample is not a testis tissue sample; and/or the olfactory receptor comprises OR5V1 and the tissue sample is not a testis, hypothalamus, basal ganglia hippocampal formation, amygdala, or fallopian tissue sample.

In one aspect, disclosed herein are methods of detecting a cancer in a subject of any preceding aspect, wherein the presence or overexpression of the olfactory receptor is assayed by polymerase chain reaction (PCR), quantitative PCR (qPCR), reverse transcriptase PCR, real time PCR, nucleic acid array, or protein array, western blot, Southern Blot, mass spectrometry, and/or liquid chromatography and/or wherein the presence or overexpression of the olfactory receptor is assayed by contacting the tissue sample with an antigen binding molecule that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and OR5V1 and measuring the presence and/or amount of the antigen binding molecule in the tissue (such as for example, by enzyme linked immunosorbent assays (ELISAs), enzyme linked immunospot assay (ELISpot). radioimmunoassays (RIA), radioimmune precipitation assays (RIPA), immunobead capture assays, and/or flow cytometry).

Also disclosed herein are methods of detecting a cancer of any preceding aspect, further comprising administering an antigen binding molecule that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and/or OR5V1 and/or an anticancer agent to a subject from a cancerous tissue sample has been detected.

In one aspect, disclosed herein are methods of treating, reducing, inhibiting, decreasing, ameliorating and/or preventing a cancer and/or metastasis in a subject comprising administering to the subject any of the antigen binding molecules of any preceding aspect (including, but not limited to an antigen binding molecule can comprise one or more complementarity determining regions (CDRs) as set forth in SEQ ID NO: 9, SEQ ID NO: 10, SEQ ID NO: 11, SEQ ID NO: 14, SEQ ID NO: 15, SEQ ID NO: 16, SEQ ID NO: 21, SEQ ID NO: 22, SEQ ID NO: 23, SEQ ID NO: 26, SEQ ID NO: 27, SEQ ID NO: 28, SEQ ID NO: 31, SEQ ID NO: 32, SEQ ID NO: 33, SEQ ID NO: 35, SEQ ID NO: 36, SEQ ID NO: 37, SEQ ID NO: 41, SEQ ID NO: 42, SEQ ID NO: 43, SEQ ID NO: 45, SEQ ID NO: 46, SEQ ID NO: 47, SEQ ID NO: 53, SEQ ID NO: 54, SEQ ID NO: 55, SEQ ID NO: 61, SEQ ID NO: 62, and/or SEQ ID NO: 63). For example, in one aspect, disclosed herein are methods of treating, reducing, inhibiting, decreasing, ameliorating and/or preventing a cancer and/or metastasis in a subject comprising administering to the subject one or more antigen binding molecules that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and OR5V1.

In one aspect, disclosed herein are methods of treating, reducing, inhibiting, decreasing, ameliorating and/or preventing a cancer and/or metastasis of any preceding aspect, wherein the cancer is a glioblastoma, melanoma, prostate cancer, esophageal cancer, head and neck cancer, lung cancer, ovarian, kidney, or stomach cancer; and wherein the antigen binding molecule selectively binds to OR2H1; wherein the cancer is a prostate cancer; and wherein the antigen binding molecule selectively binds to OR52R1; wherein the cancer is a breast cancer, lung cancer (including, but not limited to adenocarcinoma of the lung and squamous cell carcinoma of the lung), bladder cancer, melanoma, or ovarian cancer; and wherein the antigen binding molecule selectively binds to OR56A3; wherein the cancer is a liver cancer, kidney cancer, or lung cancer; and wherein the antigen binding molecule selectively binds to OR12D2; wherein the cancer is an acute myeloid leukemia (AML), esophageal cancer, prostate cancer, head and neck cancer, renal cancer, or stomach cancer; and wherein the antigen binding molecule selectively binds to OR2G3; wherein the cancer is a renal cancer (including, but not limited to clear cell renal cell carcinoma and papillary renal cell carcinoma), stomach cancer, lung cancer, esophageal cancer, melanoma, ovarian cancer or bladder cancer; and wherein the antigen binding molecule selectively binds to OR8B3; wherein the cancer is a liver cancer, breast cancer, ovarian cancer, or melanoma; and wherein the antigen binding molecule selectively binds to OR5V1; wherein the cancer is a renal cancer, stomach cancer, lung cancer, esophageal cancer, melanoma, ovarian cancer, or bladder cancer; and wherein the antigen binding molecule selectively binds to OR8B2; wherein the cancer is a prostate cancer; and wherein the antigen binding molecule selectively binds to OR51F1; wherein the cancer is a melanoma; and wherein the antigen binding molecule selectively binds to OR8K3; wherein the cancer is a stomach cancer, esophageal cancer, ovarian cancer, renal cancer, or melanoma; and wherein the antigen binding molecule selectively binds to OR8B4; wherein the cancer is a stomach cancer, esophageal cancer, renal cancer, prostate cancer, or head and neck cancer; and wherein the antigen binding molecule selectively binds to OR6Q1; wherein the cancer is a renal cancer, stomach cancer, lung cancer, head and neck cancer, or melanoma; and wherein the antigen binding molecule selectively binds to OR5M8.

Also disclosed herein are methods of treating, reducing, inhibiting, decreasing, ameliorating and/or preventing a cancer and/or metastasis of any preceding aspect, further comprising obtaining a tissue sample of a suspected cancerous tissue from the subject and assaying for the presence or overexpression of an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and/or OR5V1; wherein the presence of the olfactory receptor in the tissue sample not present in a negative control tissue sample or overexpression of the olfactory receptor in the tissue sample relative to a negative control tissue sample indicates the presence of a cancer in the subject.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows OR2H1 expression in cancerous tissue.

FIG. 1B shows OR2H1 expression in healthy tissue.

FIG. 2A shows OR52R1 expression in cancerous tissue.

FIG. 2B shows OR52R1 expression in healthy tissue.

FIG. 3A shows OR56A3 expression in cancerous tissue.

FIG. 3B shows OR56A3 expression in healthy tissue.

FIG. 4A shows OR12D2 expression in cancerous tissue.

FIG. 4B shows OR12D2 expression in healthy tissue.

FIG. 5A shows OR2G3 expression in cancerous tissue.

FIG. 5B shows OR2G3 expression in healthy tissue.

FIG. 6A shows OR8B3 expression in cancerous tissue.

FIG. 6B shows OR8B3 expression in healthy tissue.

FIG. 7A shows OR5V1 expression in cancerous tissue.

FIG. 7B shows OR5V1 expression in healthy tissue.

FIG. 7C shows OR5V1 expression in healthy tissue, High Grade Serous Ovarian Cancer (HGSOC) tissue, and breast cancer tissue.

FIG. 7D shows OR5V1 expression in non-High Grade Serous Ovarian Cancer (HGSOC) tissue.

FIG. 8 shows OR2H1 expression in healthy tissue relative to GAPDH.

FIG. 9 shows Cytotoxicity assays using luciferase-labeled lung cancer cells (top) and healthy cells (bottom). Specific cytotoxic killing of cancer cells by T cells retrovirally transduced with the OR5V1 CAR and OR2H1 CAR.

FIG. 10 shows OR2H1 expression in established cancer cell lines and cultured primary tumors (good correlation between mRNA and WB)

FIG. 11 shows in vivo experiment #2, n=5 NSG mice/group 2 experiments with comparable results. OR2H1 CAR T cells prevent tumor growth in vivo; Arrow=CAR T cell injection.

FIG. 12 shows OR2H1-specific CAR T cells also target tumor cells with low OR2H1 abundance (OR2H1^{low} luciferase-transduced OVCAR3 ovarian cancer cells).

FIG. 13 shows OR5V1 Expression in tumor cell lines and cultured primary tumors (good correlation between mRNA and WB).

FIG. 14 shows in vivo experiment #2, n=5 NSG mice/group 2 experiments with comparable results. OR5V1 CAR T cells prevent tumor growth in vivo; Arrow=CAR T cell injection

FIGS. 15A, 15B, 15C, 15D, and 15E show that OR2H1 has a limited pattern of expression in in human healthy tissues and is expressed in a variety of epithelial cancers. Real time quantitative PCR (RT-QPCR) of OR2H1 expression in (15A) human healthy tissues, (15B) High-grade serous ovarian cancer (HGSOC), (15C) Non-small cell lung cancer (NSCLC), and (15D) breast cancer. FIG. 15E shows OR2H1 expression in established cancer cell lines and cultured primary tumors via RT-QPCR demonstrates good correlation between mRNA and protein.

FIGS. 16A, 16B, 16C, 16D, and 16E show that OR5V1 has a limited pattern of expression in in human healthy tissues and is expressed in a variety of epithelial cancers. Real time quantitative PCR (RT-QPCR) of OR5V1 expression in (16A) human healthy tissues, (16B) HGSOC, (16C) NSCLC, and (16D) breast cancer. FIG. 16E shows OR5V1 expression in established cancer cell lines and cultured primary tumors via RT-QPCR demonstrates good correlation between mRNA and protein.

FIGS. 17A and 17B show that OR2H1 and OR5V1 are expressed in a variety of non-high grade serous ovarian cancer histologies. RT-QPCR of (20A) OR2H1 and (20B) OR5V1 expression in non-high grade serous ovarian cancer.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.” “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular antigen binding molecule that selectively binds OR2H1, OR52R1, OR56A3, OR12D2, OR2G3, OR8B2, OR8B3, OR51F1, OR8K3, OR8B4, OR6Q1, OR5M8, and/or OR5V1 is disclosed and discussed and a number of modifications that can be made to a number of molecules including the antigen binding molecule are discussed, specifically contemplated is each and every combination and permutation of the antigen binding molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are antigen binding molecules that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and/or OR5V1. For example, the antigen binding molecule can selectively bind to an OR5V1 olfactory receptor comprising an extracellular domain as set forth in SEQ ID NO: 5 (such as for example an OR5V1 olfactory receptor as set forth in SEQ ID NO: 4). In one aspect, the antigen binding molecule can comprise one or more complementarity determining regions (CDRs) as set forth in SEQ ID NO: 9, SEQ ID NO: 10, SEQ ID NO: 11, SEQ ID NO: 14, SEQ ID NO: 15, SEQ ID NO: 16, SEQ ID NO: 21, SEQ ID NO: 22, SEQ ID NO: 23, SEQ ID NO: 26, SEQ ID NO: 27, and/or SEQ ID NO: 28. For example, the antigen binding molecule can comprise one or more (for example, one, two, or three) complementarity determining regions (CDRs) as set forth in SEQ ID NO: 9, SEQ ID NO: 10, and SEQ ID NO: 11 (including, but not limited to an antigen binding molecule comprising the sequence as set forth in SEQ ID NO: 8); SEQ ID NO: 14, SEQ ID NO: 15, and SEQ ID NO: 16 (including, but not limited to an antigen binding molecule comprising the sequence as set forth in SEQ ID NO: 13); SEQ ID NO: 21, SEQ ID NO: 22, and SEQ ID NO: 23 (including, but not limited to an antigen binding molecule comprising the sequence as set forth in SEQ ID NO: 20); or SEQ ID NO: 26, SEQ ID NO: 27, and SEQ ID NO: 28 (including, but not limited to an antigen binding molecule comprising the sequence as set forth in SEQ ID NO: 25).

Alternatively, the antigen binding molecule can selectively bind OR2H1 olfactory receptor (such as, for example, an OR2H1 as set forth in SEQ ID NO: 1). In one aspect, the antigen binding molecule can comprise one or more complimentary determining regions (CDRs) as set forth in SEQ ID NO: 31, SEQ ID NO: 32, SEQ ID NO: 33, SEQ ID NO: 35, SEQ ID NO: 36, SEQ ID NO: 37, SEQ ID NO: 41, SEQ ID NO: 42, SEQ ID NO: 43, SEQ ID NO: 45, SEQ ID NO: 46, SEQ ID NO: 47, SEQ ID NO: 53, SEQ ID NO: 54, SEQ ID NO: 55, SEQ ID NO: 61, SEQ ID NO: 62, and/or SEQ ID NO: 63. For example, the antigen binding molecule can comprise one or more (for example, one, two, or three) complementarity determining regions (CDRs) as set forth in SEQ ID NO: 31, SEQ ID NO: 32, SEQ ID NO: 33 (including, but not limited to an antigen binding molecule comprising the sequence as set forth in SEQ ID NO: 34); SEQ ID NO: 35, SEQ ID NO: 36, and SEQ ID NO: 37 (including, but not limited to an antigen binding molecule comprising the sequence as set forth in SEQ ID NO: 38); SEQ ID NO: 41, SEQ ID NO: 42, and SEQ ID NO: 43 (including, but not limited to an antigen binding molecule comprising the sequence as set forth in SEQ ID NO: 40); SEQ ID NO: 45, SEQ ID NO: 46, and SEQ ID NO: 47 (including, but not limited to an antigen binding molecule comprising the sequence as set forth in SEQ ID NO: 44); SEQ ID NO: 53, SEQ ID NO: 54, and SEQ ID NO: 55 (including, but not limited to an antigen binding molecule comprising the sequence as set forth in SEQ ID NO: 48); and/or SEQ ID NO: 61, SEQ ID NO: 62, and SEQ ID NO: 63 (including, but not limited to an antigen binding molecule comprising the sequence as set forth in SEQ ID NO: 56). In one aspect the antigen binding molecule can comprise a variable heavy (V_{H}) chain sequence as set forth in SEQ ID NO: 65 and/or comprise a variable light (V_{L}) chain sequence as set forth in SEQ ID NO: 65.

As used herein the term “binding molecule” refers to any intact immunoglobulin including monoclonal antibodies, diabodies, polyclonal antibodies, chimeric antibodies, immunotoxins, humanized or human antibodies, as well as antibodies fragments and functional variants including antigen-binding and/or variable domain variants comprising fragment of an immunoglobulin that competes with the intact immunoglobulin for specific binding to the binding partner of the immunoglobulin, e.g. OR2H1, OR52R1, OR56A3, OR12D2, OR2G3, OR8B2, OR8B3, OR51F1, OR8K3, OR8B4, OR6Q1, OR5M8, and/or OR5V1. A binding molecule can also refer to any peptide, protein, functional nucleic acid (such as, for example, an RNAi, siRNA, antisense oligonucleotide), chimeric antigen receptor (CAR) T cells, CAR NK cells, CAR macrophage (CARMA) that can bind to a target, e.g., OR2H1, OR52R1, OR56A3, OR12D2, OR2G3, OR8B2, OR8B3, OR51F1, OR8K3, OR8B4, OR6Q1, OR5M8, and/or OR5V1. As noted above a binding molecule can be an immunotoxin which, as used herein, refers to any antibody or functional antibody fragment thereof that is linked to a toxin moiety. In one aspect, the antigen binding molecule can comprise a CAR T cell that selectively binds OR5V1 olfactory receptor and wherein the chimeric antigen receptor of the CAR T cell comprises a sequence as set forth in SEQ ID NO: 17. In another aspect, the antigen binding molecule can comprise a CAR T cell that selectively binds OR2H1 olfactory receptor and wherein the chimeric antigen receptor of the CAR T cell comprises a sequence as set forth in SEQ ID NO: 30.

1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with OR2H1, OR52R1, OR56A3, OR12D2, OR2G3, OR8B2, OR8B3, OR51F1, OR8K3, OR8B4, OR6Q1, OR5M8, and/or OR5V1. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain OR2H1, OR52R1, OR56A3, OR12D2, OR2G3, OR8B2, OR8B3, OR51F1, OR8K3, OR8B4, OR6Q1, OR5M8, and/or OR5V1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)). In one aspect, the antigen binding domain can comprise one or more (for example, one, two, or three) complementarity determining regions (CDRs) as set forth in SEQ ID NO: 9, SEQ ID NO: 10, and SEQ ID NO: 11 (including, but not limited to an antigen binding molecule comprising the sequence as set forth in SEQ ID NO: 8); SEQ ID NO: 14, SEQ ID NO: 15, and SEQ ID NO: 16 (including, but not limited to an antigen binding molecule comprising the sequence as set forth in SEQ ID NO: 13); SEQ ID NO: 21, SEQ ID NO: 22, and SEQ ID NO: 23 (including, but not limited to an antigen binding molecule comprising the sequence as set forth in SEQ ID NO: 20); or SEQ ID NO: 26, SEQ ID NO: 27, and SEQ ID NO: 28 (including, but not limited to an antigen binding molecule comprising the sequence as set forth in SEQ ID NO: 25); SEQ ID NO: 31, SEQ ID NO: 32, SEQ ID NO: 33 (including, but not limited to an antigen binding molecule comprising the sequence as set forth in SEQ ID NO: 34); SEQ ID NO: 35, SEQ ID NO: 36, and SEQ ID NO: 37 (including, but not limited to an antigen binding molecule comprising the sequence as set forth in SEQ ID NO: 38); SEQ ID NO: 41, SEQ ID NO: 42, and SEQ ID NO: 43 (including, but not limited to an antigen binding molecule comprising the sequence as set forth in SEQ ID NO: 40); SEQ ID NO: 45, SEQ ID NO: 46, and SEQ ID NO: 47 (including, but not limited to an antigen binding molecule comprising the sequence as set forth in SEQ ID NO: 44); SEQ ID NO: 53, SEQ ID NO: 54, and SEQ ID NO: 55 (including, but not limited to an antigen binding molecule comprising the sequence as set forth in SEQ ID NO: 48); and/or SEQ ID NO: 61, SEQ ID NO: 62, and SEQ ID NO: 63 (including, but not limited to an antigen binding molecule comprising the sequence as set forth in SEQ ID NO: 56). In one aspect the antigen binding molecule can comprise a variable heavy (V_{H}) chain sequence as set forth in SEQ ID NO: 65 and/or comprise a variable light (V_{L}) chain sequence as set forth in SEQ ID NO: 65.

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti-OR2H1, OR52R1, OR56A3, OR12D2, OR2G3, OR8B2, OR8B3, OR51F1, OR8K3, OR8B4, OR6Q1, OR5M8, and/or OR5Vlantibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable..

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

3. Homology/Identity

It is understood that one way to define any known variants and derivatives or those that might arise, of the disclosed genes and proteins herein is through defining the variants and derivatives in terms of homology to specific known sequences. For example, SEQ ID NO: 1 sets forth a particular sequence of an OR2H1 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### C. Methods of Detecting a Cancer

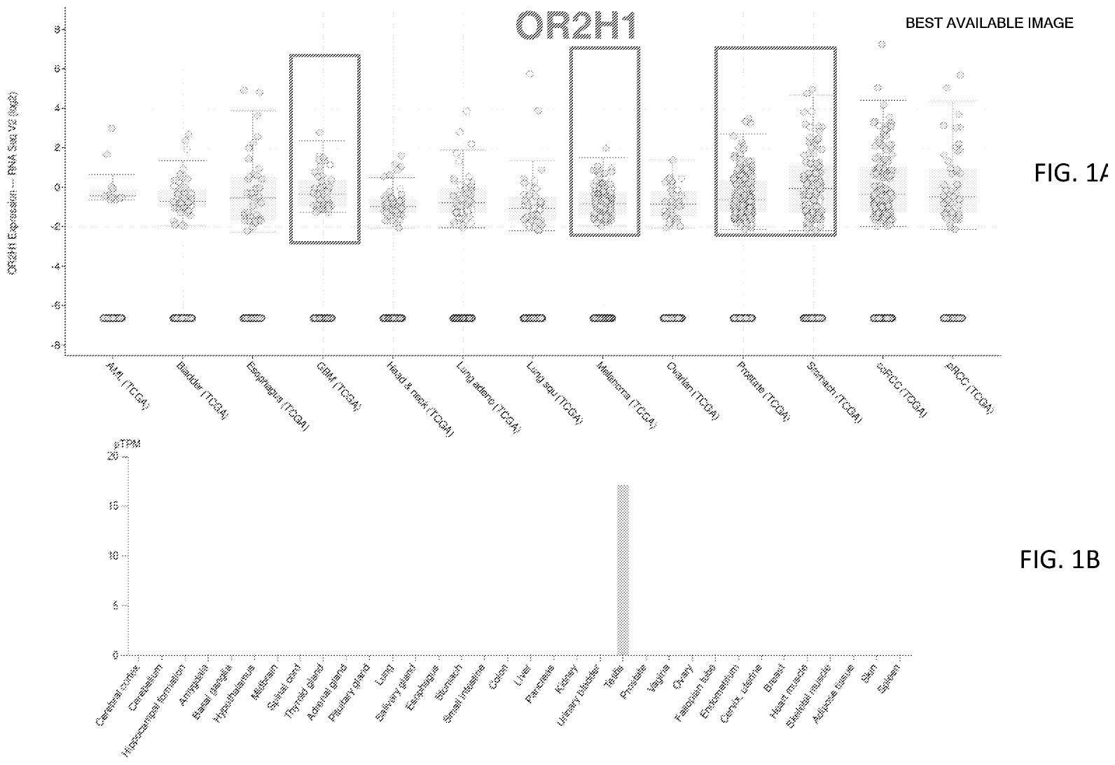

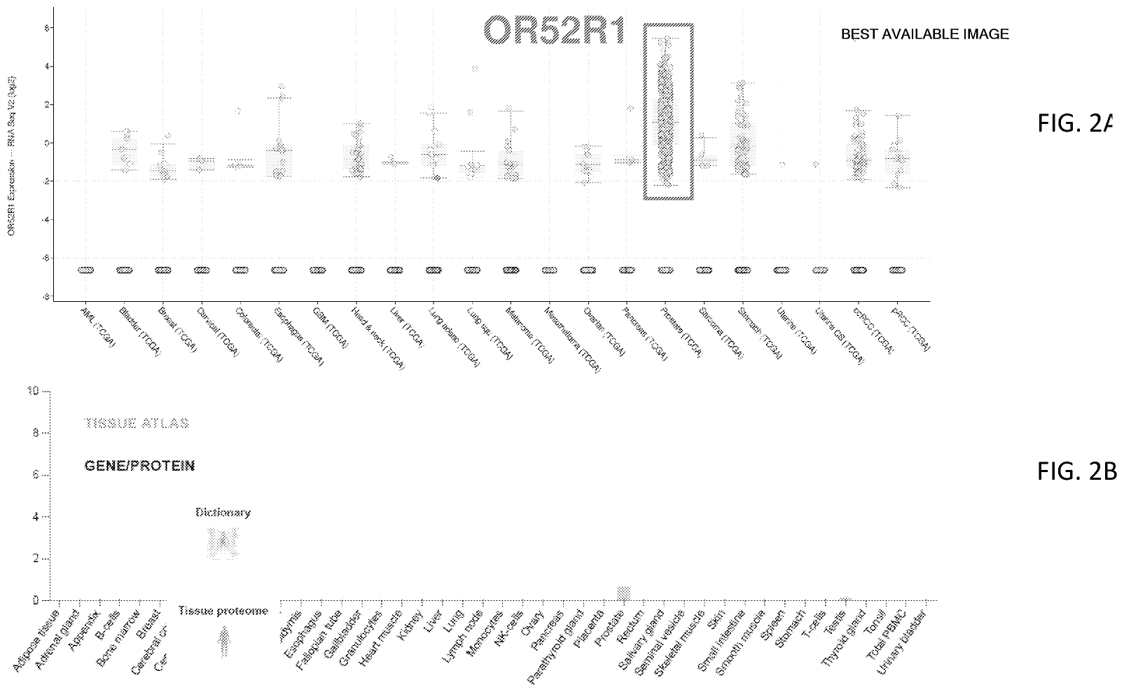

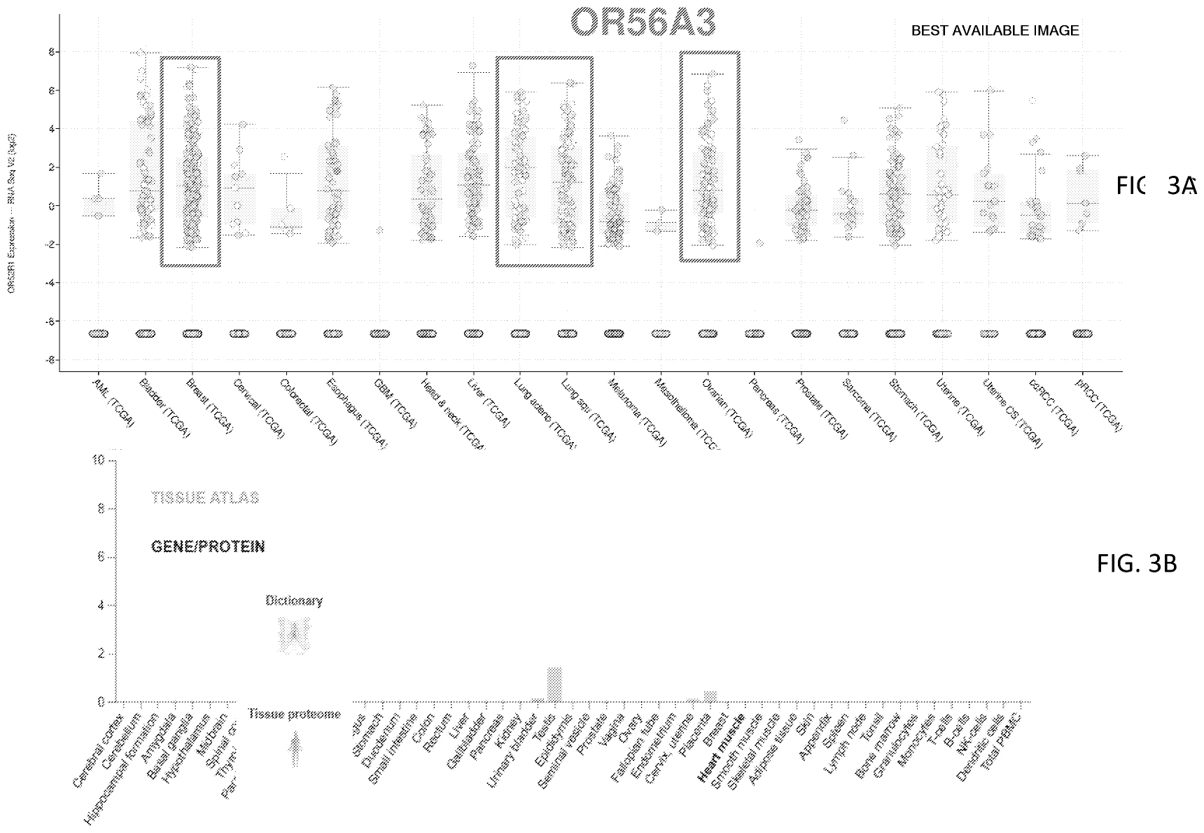

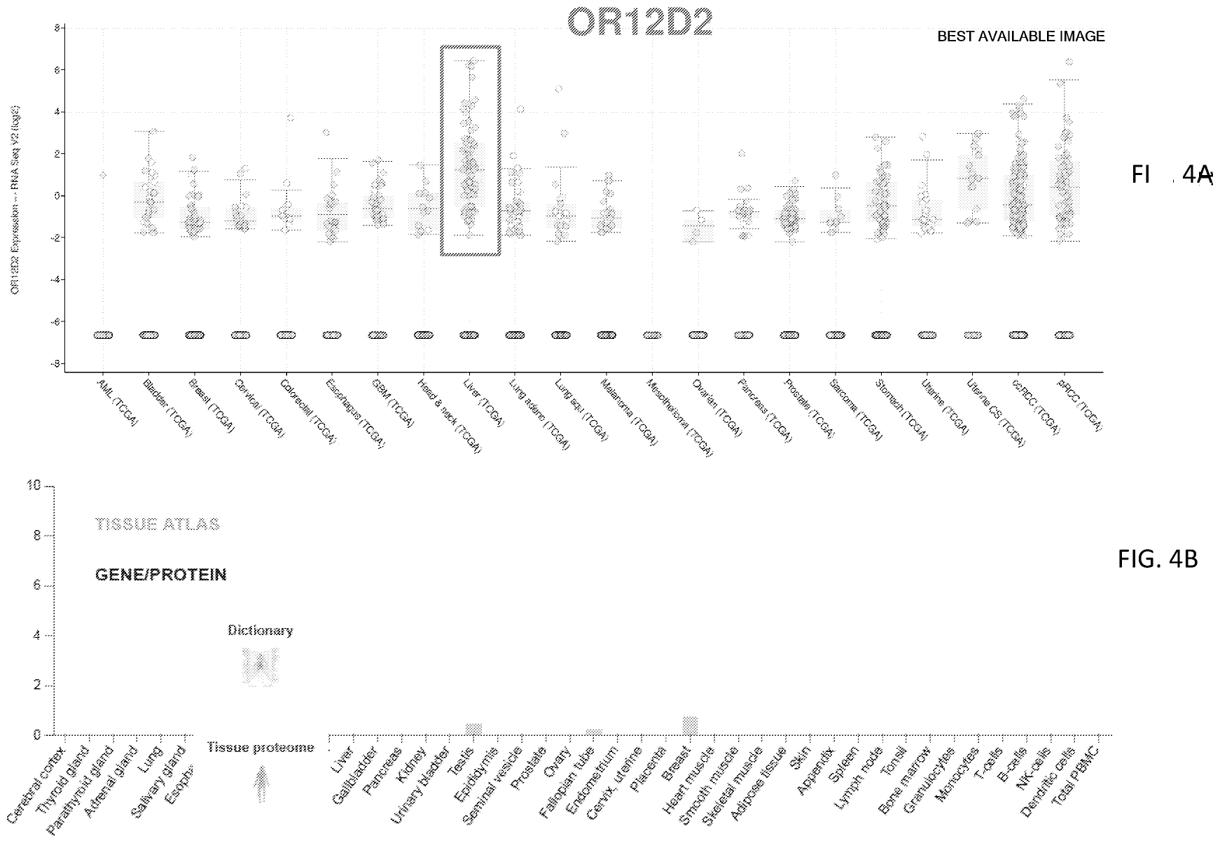

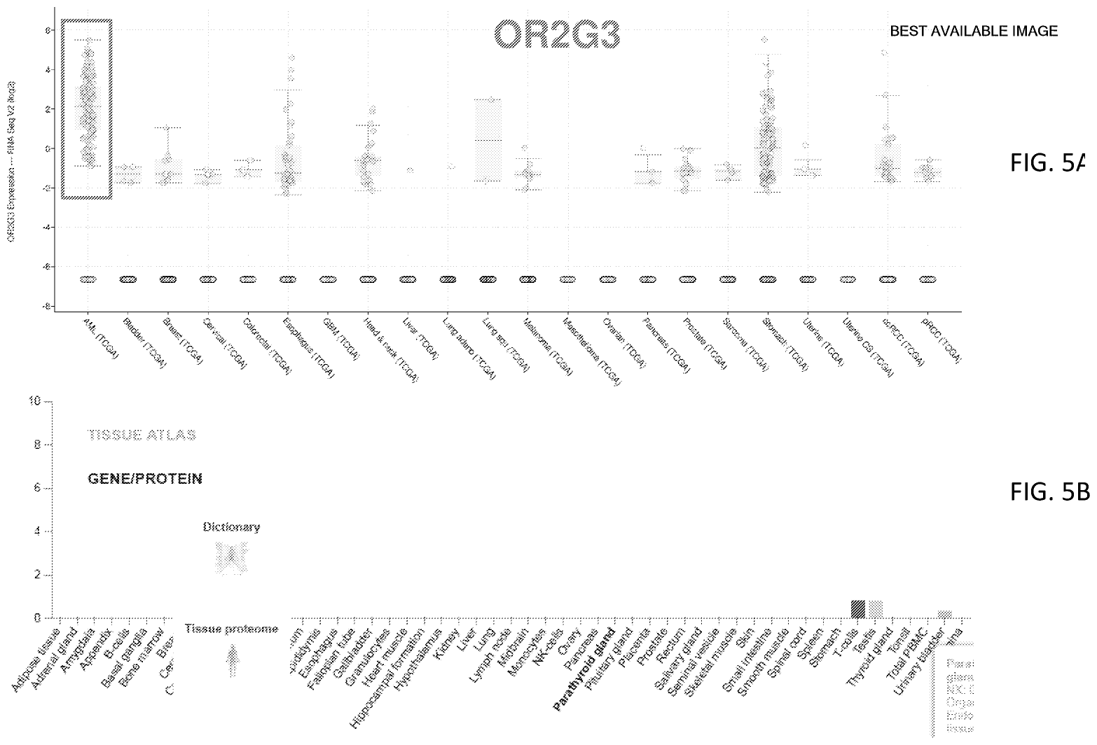

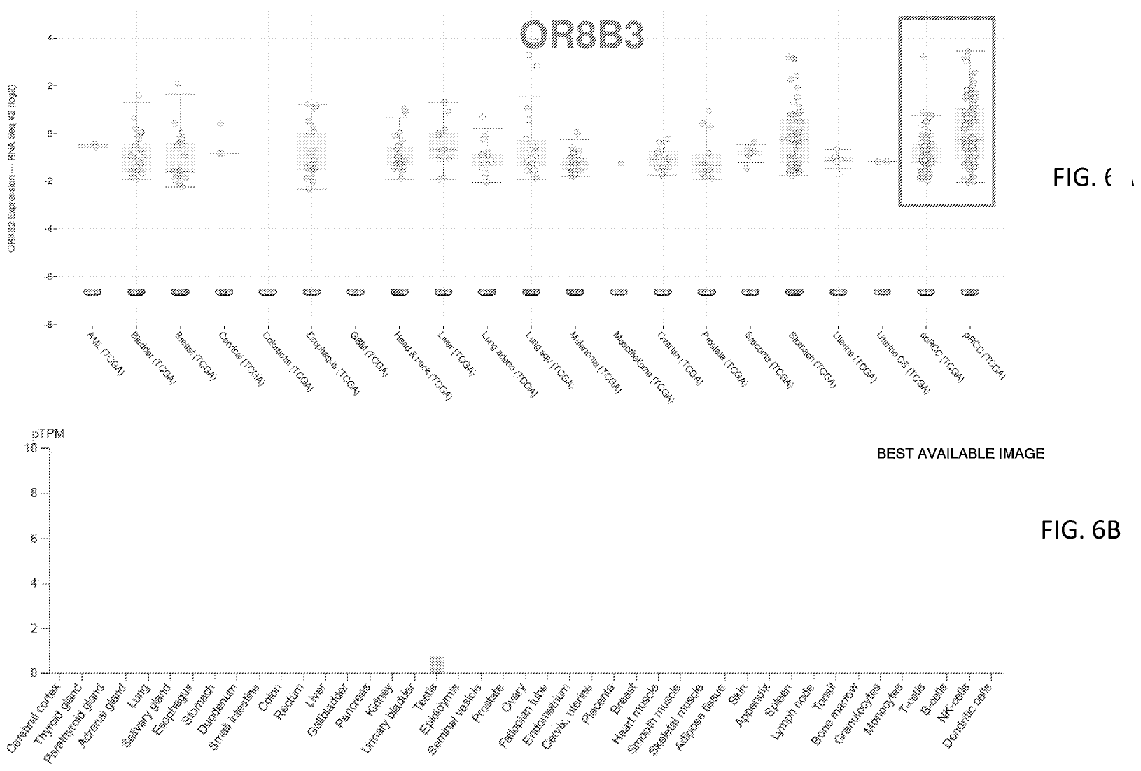

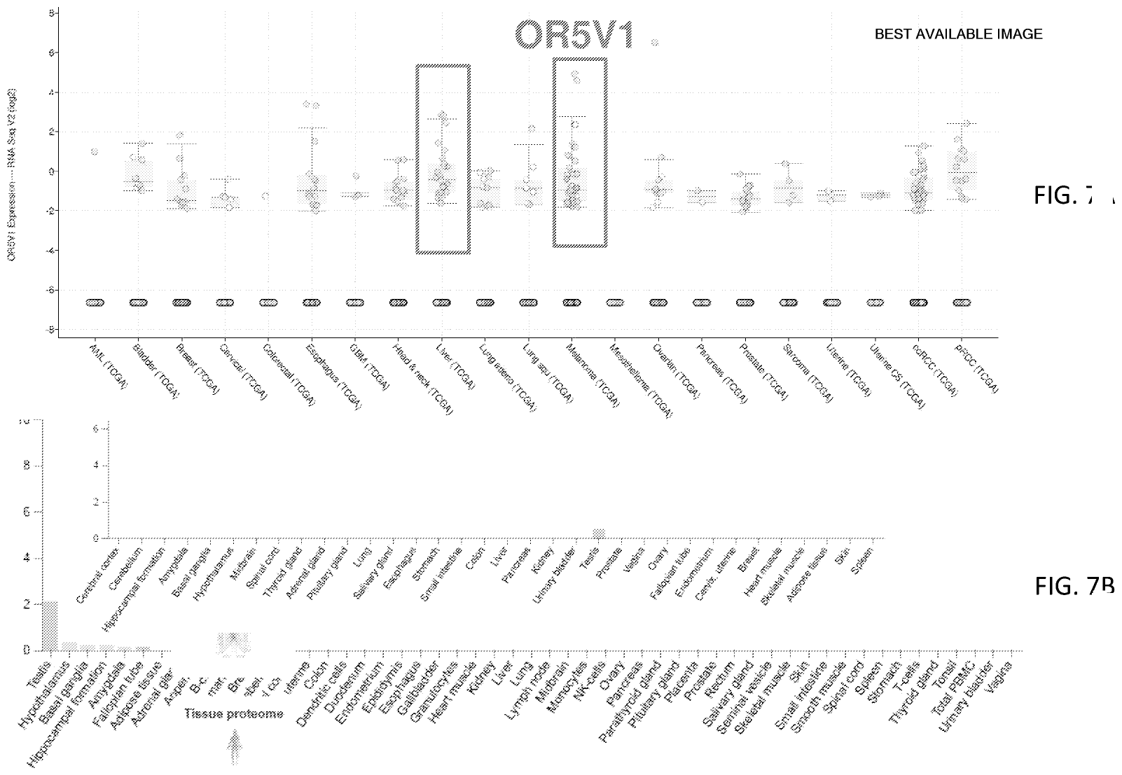

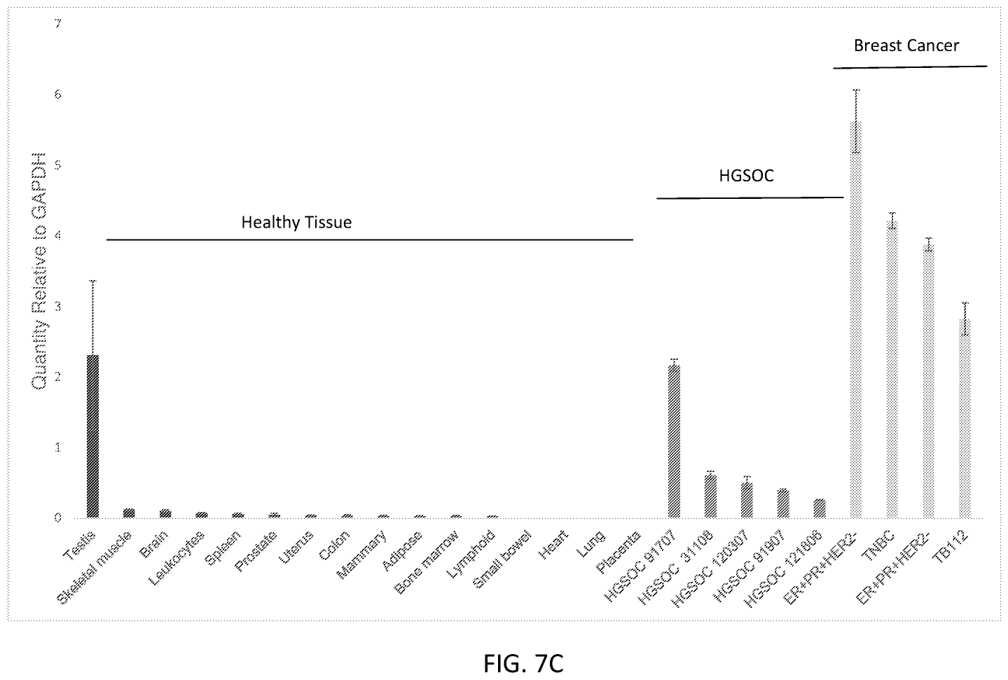

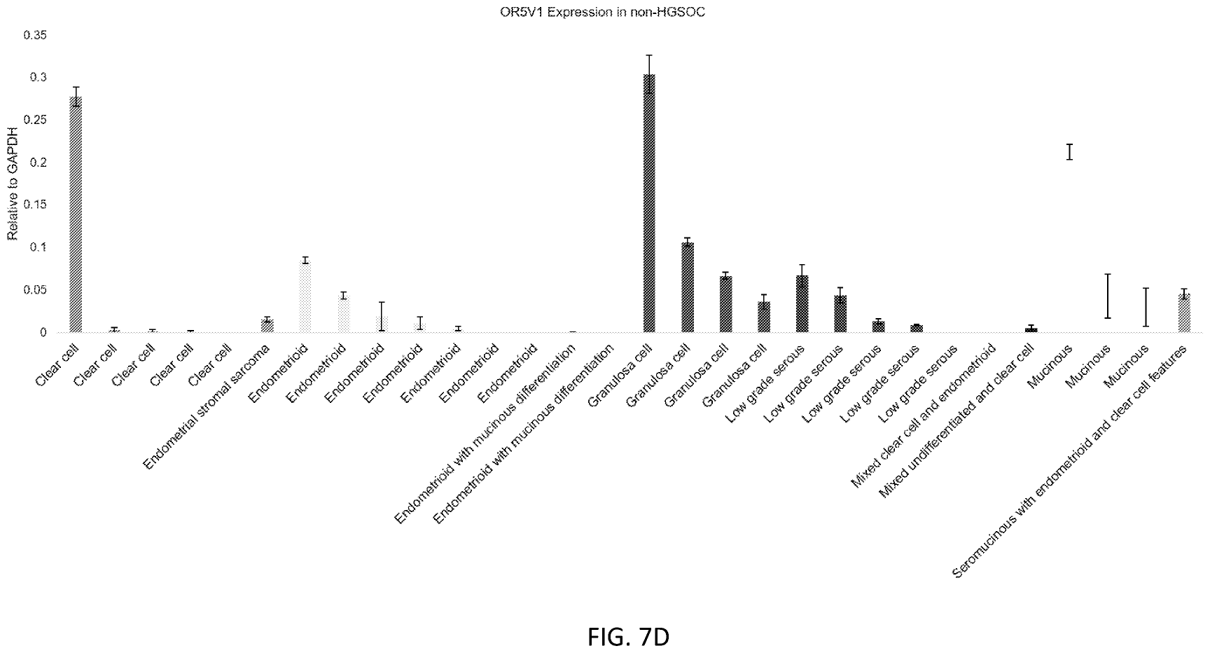

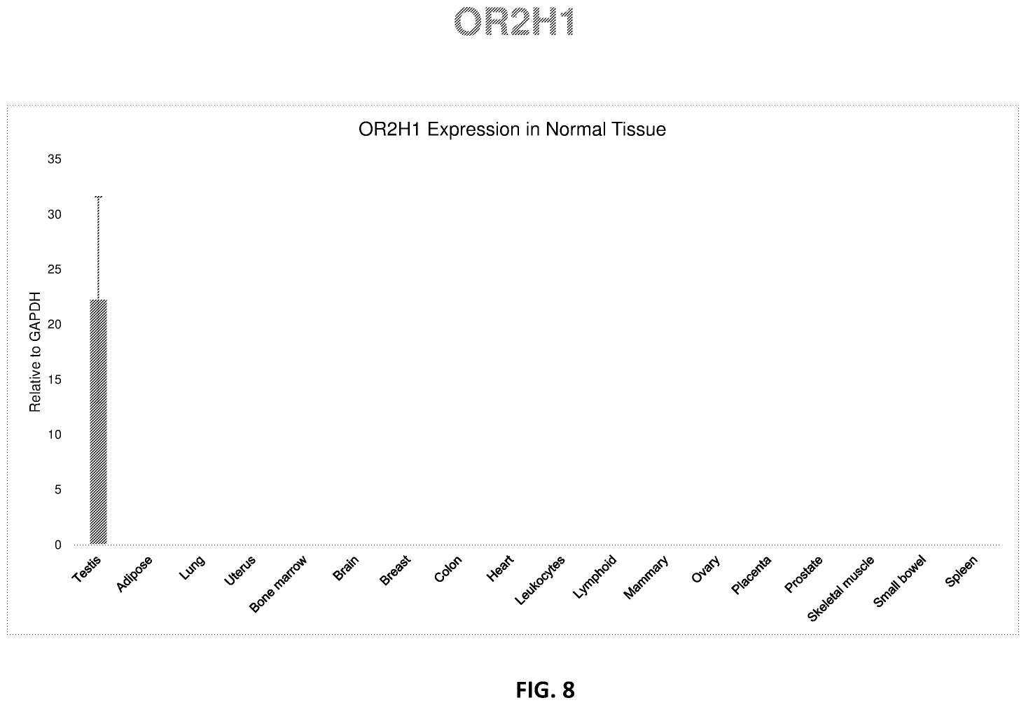

As shown herein, the olfactory receptors OR2H1, OR52R1, OR56A3, OR12D2, OR2G3, OR8B2, OR8B3, OR51F1, OR8K3, OR8B4, OR6Q1, OR5M8, and OR5V1 while normally expressed in some tissues are overexpressed or aberrantly expressed in other tissue (see for example FIGS. 1-8). Thus, it is understood that the presence and/or overexpression of OR2H1, OR52R1, OR56A3, OR12D2, OR2G3, OR8B2, OR8B3, OR51F1, OR8K3, OR8B4, OR6Q1, OR5M8, and/or OR5V1 can be used as a biomarker to detect and/or diagnose a cancer in a subject. Accordingly, in one aspect, disclosed herein are methods of detecting a cancer in a subject comprising obtaining a tissue sample of a suspected cancerous tissue from the subject and contacting the tissue sample with the antigen binding molecule of any preceding aspect. For example, disclosed herein, in one aspect, are methods of detecting a cancer in a subject comprising obtaining a tissue sample of a suspected cancerous tissue from the subject and assaying for the presence or overexpression of an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and OR5V1; wherein the presence of the olfactory receptor in the tissue sample not present in a negative control tissue sample or overexpression of the olfactory receptor in the tissue sample relative to a negative control tissue sample indicates the presence of a cancer in the subject. The use of a negative control insures that the detection method does not create a false positive by detecting the presence of an olfactory receptor already present in normal tissue or providing a basis for determining whether the amount of the olfactory receptor expressed in the subject's tissue sample is higher (i.e. overexpressed) relative to normal tissue.

It is understood and herein contemplated that expression in a tissue alone is not always sufficient for the detection/diagnosis of a cancer as some tissues normally express OR2H1, OR52R1, OR56A3, OR12D2, OR2G3, OR8B2, OR8B3, OR51F1, OR8K3, OR8B4, OR6Q1, OR5M8, and OR5V1. Thus, in one aspect, disclosed herein are methods of detecting and/or diagnosing a cancer in a subject wherein the olfactory receptor comprises OR2H1 and the tissue sample is not a testis tissue sample; the olfactory receptor comprises OR52R1 and the tissue sample is not a prostate tissue sample; the olfactory receptor comprises OR56A3 and the tissue sample is not a urinary bladder, testis, cervix or placenta tissue sample; the olfactory receptor comprises OR12D2 and the tissue sample is not a testis, fallopian tube, or breast tissue sample; the olfactory receptor comprises OR2G3 and the tissue sample is not a testis, T cell, or urinary bladder tissue sample; the olfactory receptor comprises OR8B3 and the tissue sample is not a testis tissue sample; and/or the olfactory receptor comprises OR5V1 and the tissue sample is not a testis, hypothalamus, basal ganglia hippocampal formation, amygdala, or fallopian tissue sample. For example, disclosed herein are methods of detecting and/or diagnosing a cancer in a subject wherein the olfactory receptor comprises OR5V1 and the antigen binding molecule comprises one or more (for example, one, two, or three) complementarity determining regions (CDRs) as set forth in SEQ ID NO: 9, SEQ ID NO: 10, and SEQ ID NO: 11 (including, but not limited to an antigen binding molecule comprising the sequence as set forth in SEQ ID NO: 8); SEQ ID NO: 14, SEQ ID NO: 15, and SEQ ID NO: 16 (including, but not limited to an antigen binding molecule comprising the sequence as set forth in SEQ ID NO: 13); SEQ ID NO: 21, SEQ ID NO: 22, and SEQ ID NO: 23 (including, but not limited to an antigen binding molecule comprising the sequence as set forth in SEQ ID NO: 20); or SEQ ID NO: 26, SEQ ID NO: 27, and SEQ ID NO: 28 (including, but not limited to an antigen binding molecule comprising the sequence as set forth in SEQ ID NO: 25). Alternatively, disclosed herein are methods of detecting and/or diagnosing a cancer in a subject, wherein the olfactory receptor comprises OR2H1 and the antigen binding molecule comprises one or more (for example, one, two, or three) complementarity determining regions (CDRs) as set forth in SEQ ID NO: 31, SEQ ID NO: 32, SEQ ID NO: 33 (including, but not limited to an antigen binding molecule comprising the sequence as set forth in SEQ ID NO: 34); SEQ ID NO: 35, SEQ ID NO: 36, and SEQ ID NO: 37 (including, but not limited to an antigen binding molecule comprising the sequence as set forth in SEQ ID NO: 38); SEQ ID NO: 41, SEQ ID NO: 42, and SEQ ID NO: 43 (including, but not limited to an antigen binding molecule comprising the sequence as set forth in SEQ ID NO: 40); SEQ ID NO: 45, SEQ ID NO: 46, and SEQ ID NO: 47 (including, but not limited to an antigen binding molecule comprising the sequence as set forth in SEQ ID NO: 44); SEQ ID NO: 53, SEQ ID NO: 54, and SEQ ID NO: 55 (including, but not limited to an antigen binding molecule comprising the sequence as set forth in SEQ ID NO: 48); and/or SEQ ID NO: 61, SEQ ID NO: 62, and SEQ ID NO: 63 (including, but not limited to an antigen binding molecule comprising the sequence as set forth in SEQ ID NO: 56). In one aspect the antigen binding molecule can comprise a variable heavy (V_{H}) chain sequence as set forth in SEQ ID NO: 65 and/or comprise a variable light (V_{L}) chain sequence as set forth in SEQ ID NO: 65.

The detection methods can employ any chemical, radiological, microscopy, immunological or molecular biological method or technique for detecting the presence or amount of a protein or RNA in a tissue. In one aspect, disclosed herein are methods of detecting a cancer in a subject, wherein the presence or overexpression of the olfactory receptor is assayed by polymerase chain reaction (PCR), quantitative PCR (qPCR), reverse transcriptase PCR, real time PCR, nucleic acid array, or protein array, western blot, Southern Blot, mass spectrometry, and/or liquid chromatography and/or wherein the presence or overexpression of the olfactory receptor is assayed by contacting the tissue sample with an antigen binding molecule that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and OR5V1 and measuring the presence and/or amount of the antigen binding molecule in the tissue (such as for example, by enzyme linked immunosorbent assays (ELISAs), enzyme linked immunospot assay (ELISpot). radioimmunoassays (RIA), radioimmune precipitation assays (RIPA), immunobead capture assays, and/or flow cytometry).

1. Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow IOGF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, NJ) and specialised chip designs, such as engineered microchannels in a plate (e.g., The Living Chip™, Biotrove, Woburn, MA) and tiny 3D posts on a silicon surface (Zyomyx, Hayward CA). Particles in suspension can also be used as the basis of arrays, providing they are coded for identification; systems include colour coding for microbeads (Luminex, Austin, TX; Bio-Rad Laboratories) and semiconductor nanocrystals (e.g., QDots™, Quantum Dot, Hayward, CA), and barcoding for beads (UltraPlex™, SmartBead Technologies Ltd, Babraham, Cambridge, UK) and multimetal microrods (e.g., Nanobarcodes™ particles, Nanoplex Technologies, Mountain View, CA). Beads can also be assembled into planar arrays on semiconductor chips (LEAPS technology, BioArray Solutions, Warren, NJ).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, WA) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, MA), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), rolling circle DNA amplification (Molecular Staging, New Haven CT), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, CA; Clontech, Mountain View, CA; BioRad; Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, CA; Biosite, San Diego, CA). In addition to the conventional antibodies, Fab and scFv fragments, single V-domains from camelids or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, CO). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, CA).

Another methodology which can be used diagnostically and in expression profiling is the ProteinChip® array (Ciphergen, Fremont, CA), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, CT).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

It is understood and herein contemplated that once a cancer is detected and/or diagnosed, the next step would be to treat said cancer. Accordingly, disclosed herein are methods of detecting a caner of any preceding aspect, further comprising administering an antigen binding molecule that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and/or OR5V1 and/or an anti-cancer agent to a subject from a cancerous tissue sample has been detected.

### D. Method of Treating Cancer

As noted above, the disclosed olfactory receptors (i.e., OR2H1, OR52R1, OR56A3, OR12D2, OR2G3, OR8B2, OR8B3, OR51F1, OR8K3, OR8B4, OR6Q1, OR5M8, and OR5V1) are only present or are overexpressed in cancerous tissue and can serve as biomarkers for detection and/or diagnosis of a cancer. It is further understood that once detected, these same biomarkers can serve as targets for the treatment of the cancer. Thus, in one aspect, disclosed herein are methods of treating, reducing, inhibiting, decreasing, ameliorating and/or preventing a cancer and/or metastasis in a subject comprising administering to the subject any of the antigen binding molecules disclosed herein. For example, in one aspect, disclosed herein are methods of treating, reducing, inhibiting, decreasing, ameliorating and/or preventing a cancer and/or metastasis in a subject comprising administering to the subject one or more antigen binding molecules that selective binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and/or OR5V1.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, acute myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, epithelial cancer, renal cancer (including, but not limited to, clear cell renal cell carcinoma and papillary renal cell carcinoma), genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

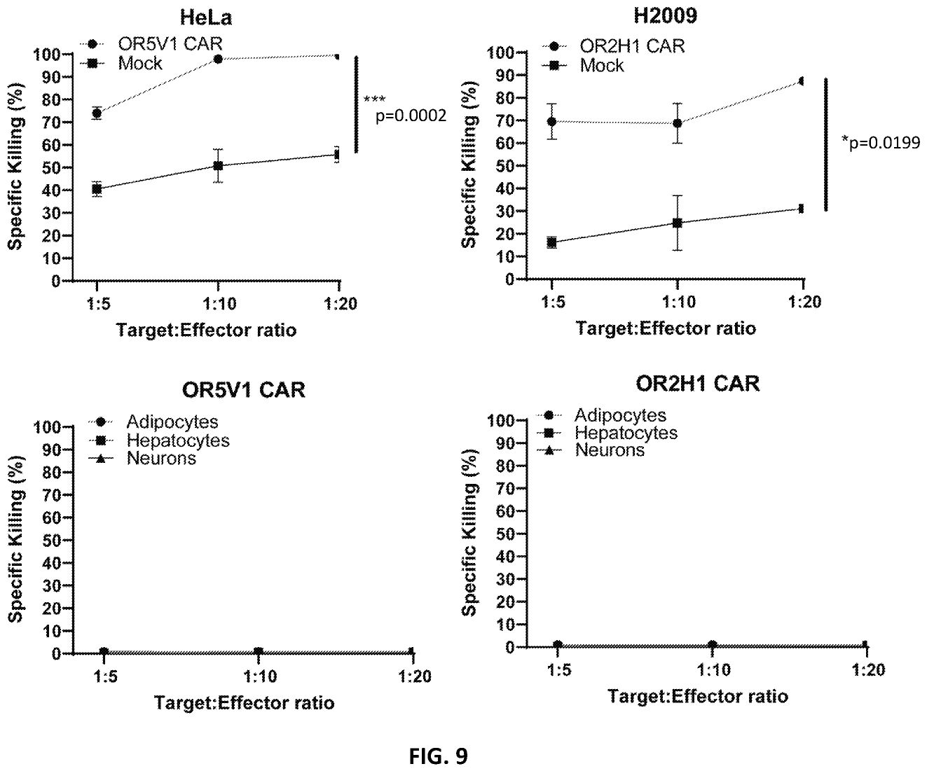

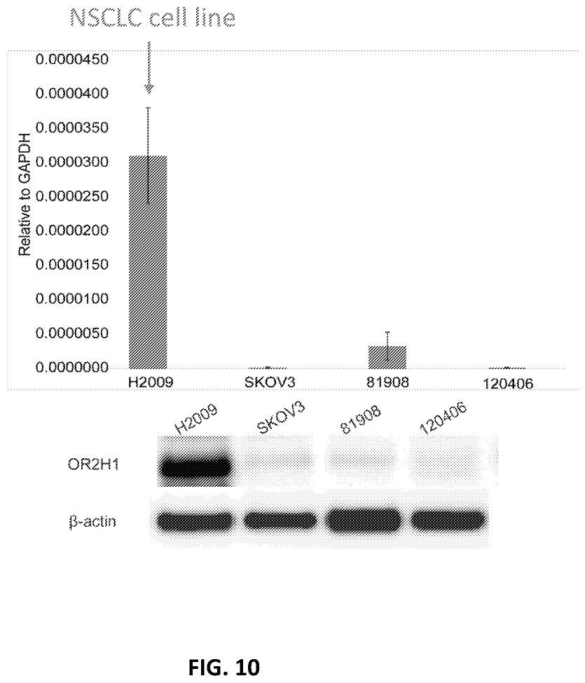

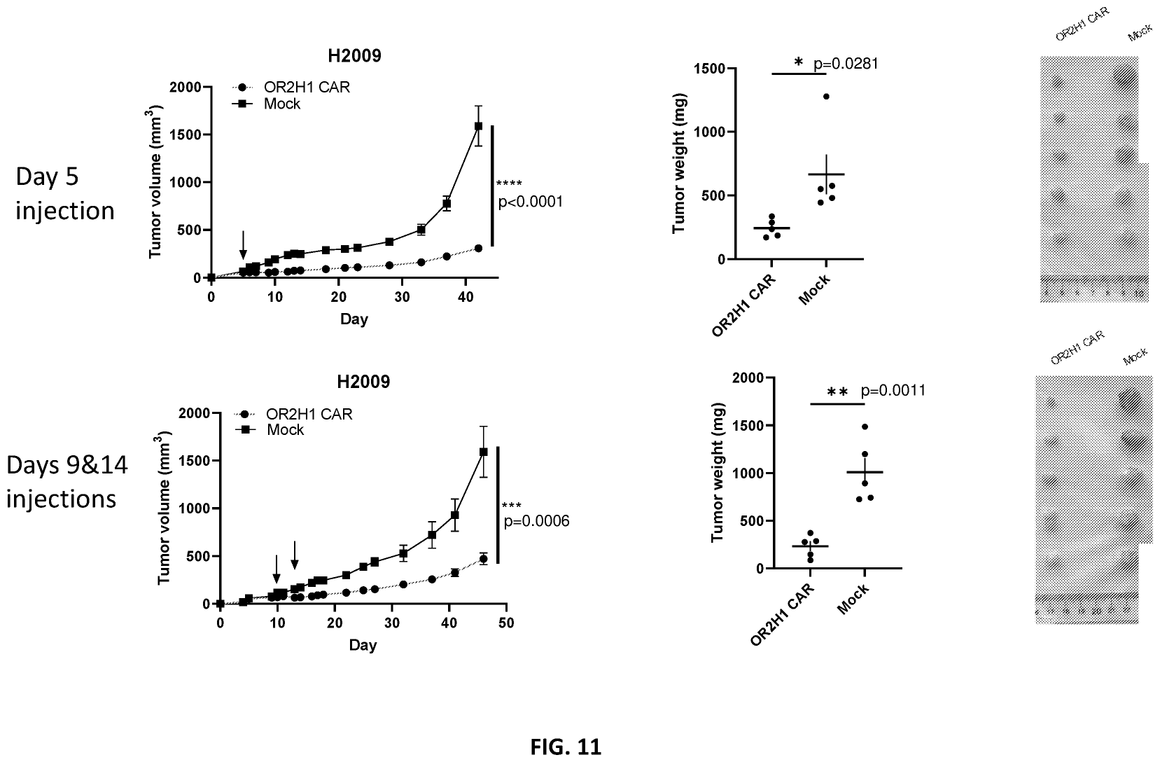

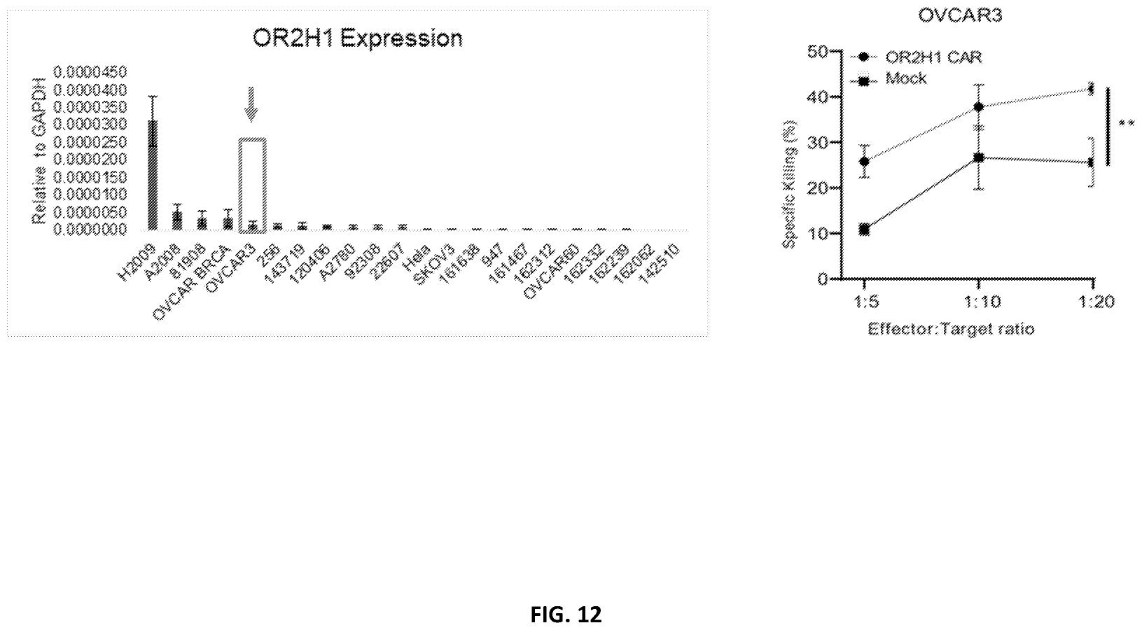

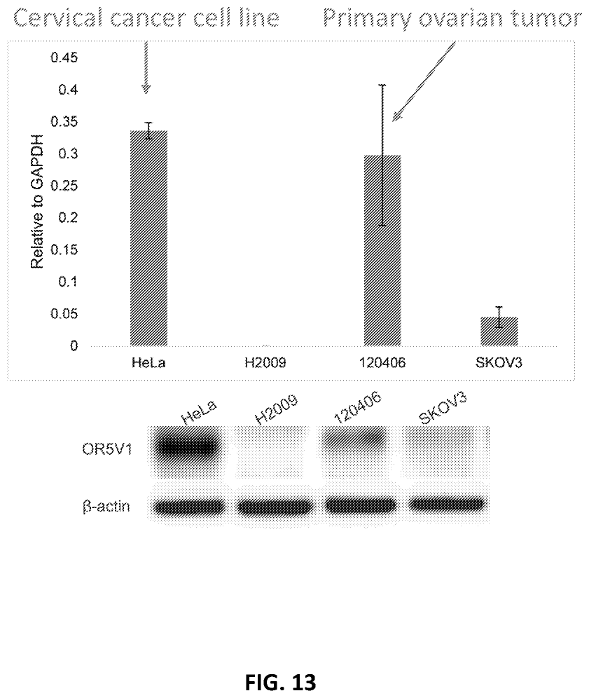

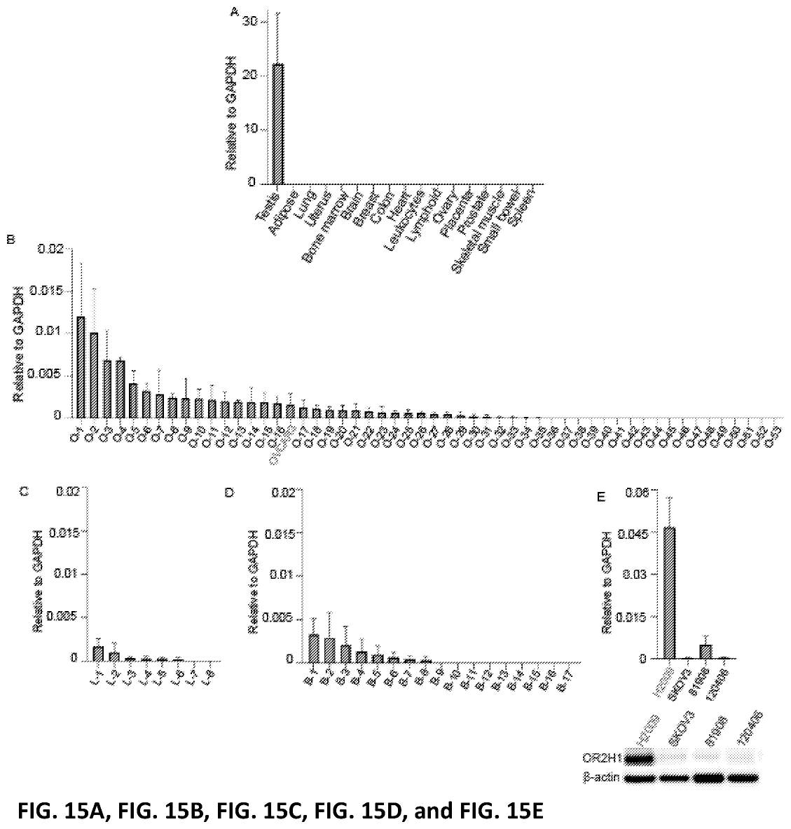

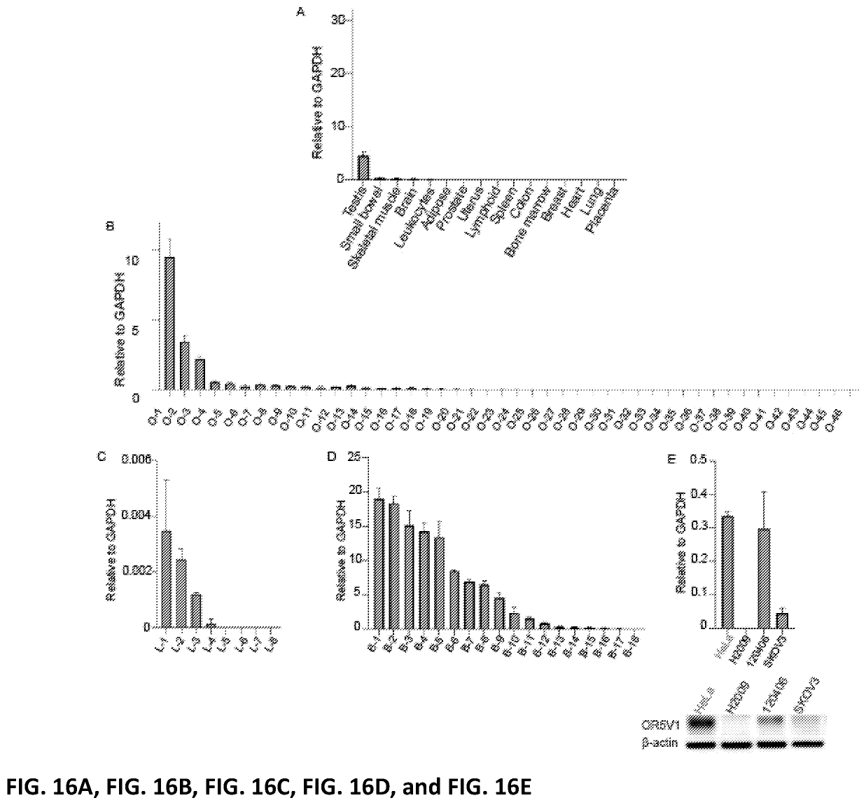

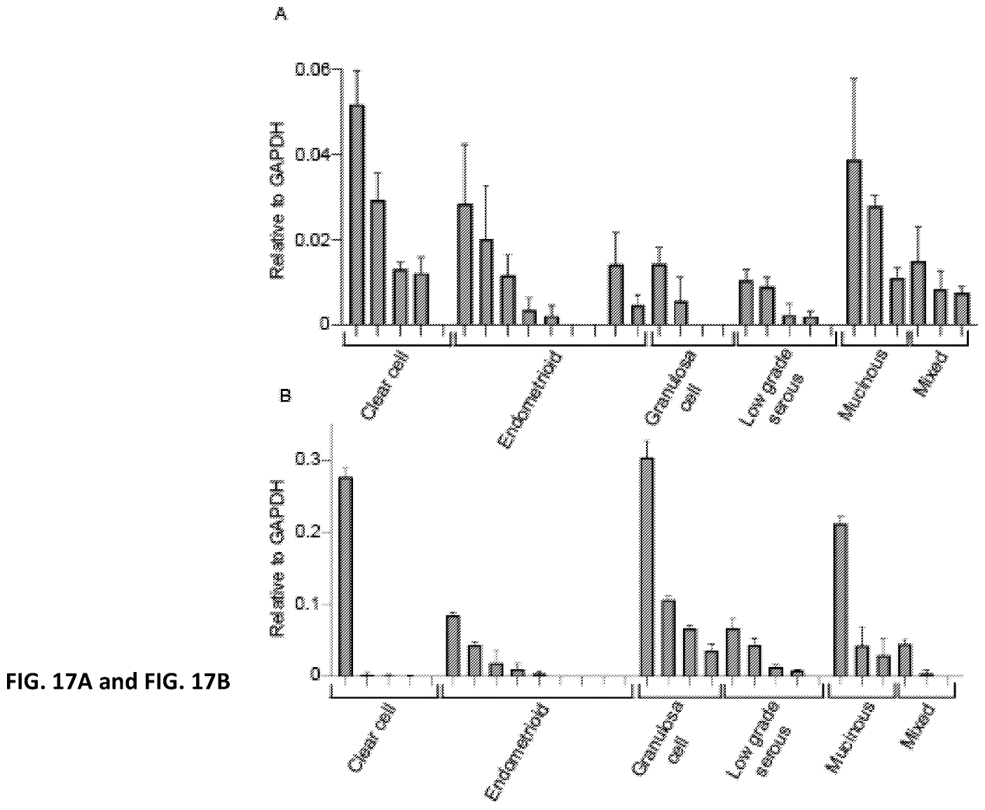

It is understood and herein contemplated that not every olfactory receptor is expressed in every cancer and not every cancer expresses an olfactory receptor. As shown in FIGS. 15-17, expression of OR2H1 and OR5V1 have limited expression in healthy tissue, but are expressed in cancers. Thus, as evidenced by FIGS. 9-14, the olfactory receptors can serve as cytotoxic targets in cancer cells. Specifically, FIGS. 9, 11, and 12 show that OR2H1 can be effectively targeted to treat at least NSCLC and HGSOC through the use of chimeric antigen receptors without cytotoxicity of normal healthy tissues. FIGS. 9 and 12 show the cytotoxicity of OR2H1 CAR or mock transduced T-cells of H2009-luciferase (FIG. 9) and OVCAR3-luciferase (FIG. 12) tumors measured by luciferase detection after co-culture of 8 hours. FIG. 11 shows tumor volume, tumor weight, and tumors of H2009 grown in the flank of NOD-SCID mice (n=5 per group; 2 independent experiments with comparable results) after treatment with a single retro-orbital injection of 5 million OR2H1 CAR or mock transduced T cells 5 days after tumor cell injection (arrow). FIG. 9 also shows the cytotoxicity of OR2H1 CAR or mock transduced T-cells of normal adipocytes, hepatocytes, and neurons transfected with luciferase measured by luciferase detection after co-culture of 8 hours.

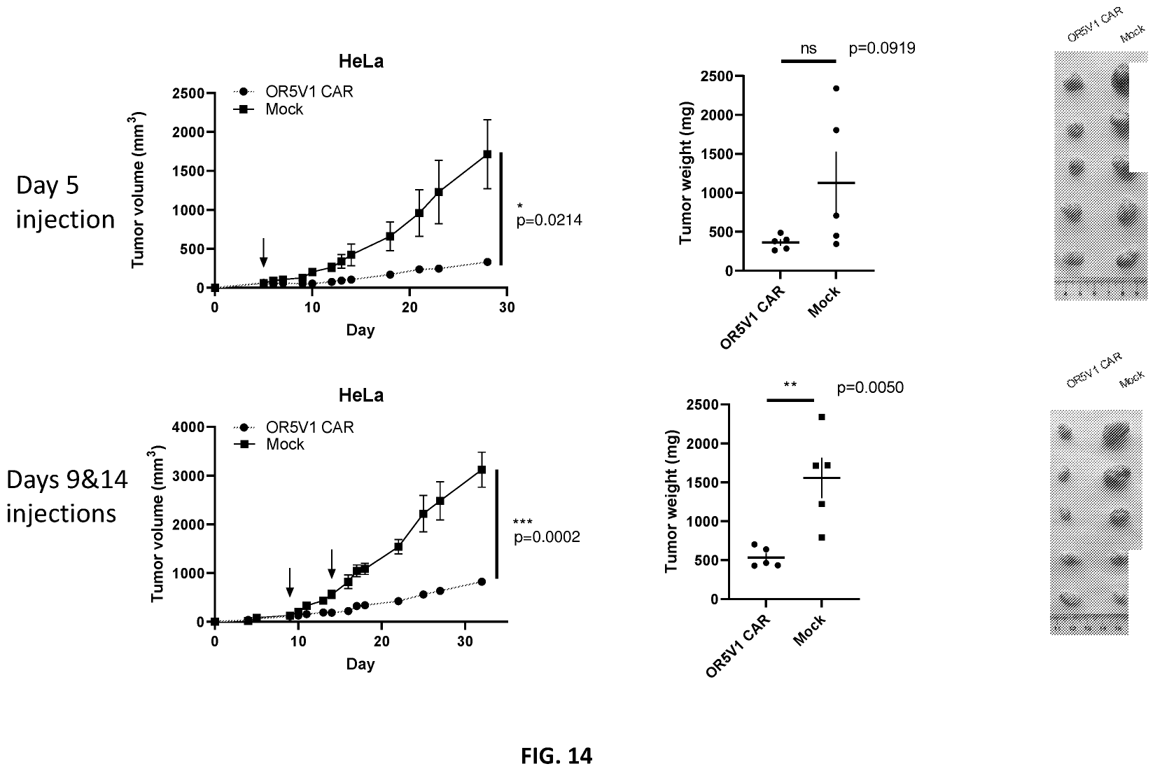

Additionally, FIGS. 9 and 14 show that OR5V1 can be effectively targeted to treat at least cervical cancer through the use of chimeric antigen receptors without cytotoxicity of normal healthy tissues. FIG. 9 shows the cytotoxicity of OR5V1 CAR or mock transduced T-cells of HeLa-luciferase tumors measured by luciferase detection after co-culture of 8 hours. FIG. 14 shows tumor volume, tumor weight, and tumors of HeLa grown in the flank of NOD-SCID mice (n=5 per group; 2 independent experiments with comparable results) after treatment with a single retro-orbital injection of 5 million OR5V1 CAR or mock transduced T cells 5 days after tumor cell injection (arrow). FIG. 9 also shows cytotoxicity of OR5V1 CAR or mock transduced T-cells of normal adipocytes, hepatocytes, and neurons transfected with luciferase measured by luciferase detection after co-culture of 8 hours.

Thus, in one aspect, disclosed herein are methods of treating, reducing, inhibiting, decreasing, ameliorating and/or preventing a cancer and/or metastasis, wherein the cancer is a glioblastoma, melanoma, prostate cancer, esophageal cancer, head and neck cancer, lung cancer, ovarian, kidney, or stomach cancer; and wherein the antigen binding molecule selectively binds to OR2H1; wherein the cancer is a prostate cancer; and wherein the antigen binding molecule selectively binds to OR52R1; wherein the cancer is a breast cancer, lung cancer, bladder cancer, melanoma, or ovarian cancer; and wherein the antigen binding molecule selectively binds to OR56A3; wherein the cancer is a lung cancer selected from lung adenocarcinoma and lung squamous cell carcinoma; wherein the cancer is a liver cancer, kidney cancer, or lung cancer; and wherein the antigen binding molecule selectively binds to OR12D2; wherein the cancer is an acute myeloid leukemia (AML), esophageal cancer, prostate cancer, head and neck cancer, renal cancer, or stomach cancer; and wherein the antigen binding molecule selectively binds to OR2G3; wherein the cancer is a renal cancer, stomach cancer, lung cancer, esophageal cancer, melanoma, ovarian cancer or bladder cancer; and wherein the antigen binding molecule selectively binds to OR8B3; wherein the renal cancer is selected from clear cell renal cell carcinoma and papillary renal cell carcinoma; wherein the cancer is a liver cancer, breast cancer (including triple negative breast cancer), ovarian cancer (including, but not limited to high High Grade Serous Ovarian Cancer (HGSOC)), or melanoma; and wherein the antigen binding molecule selectively binds to OR5V1 (for example, an OR5V1 olfactory receptor comprising an extracellular domain of an olfactory receptor OR5V1 as set forth in SEQ ID NO: 5 (including, but not limited to an OR5V1 as set forth in SEQ ID NO: 4)); wherein the cancer is a renal cancer, stomach cancer, lung cancer, esophageal cancer, melanoma, ovarian cancer, or bladder cancer; and wherein the antigen binding molecule selectively binds to OR8B2; wherein the cancer is a prostate cancer; and wherein the antigen binding molecule selectively binds to OR51F1; wherein the cancer is a melanoma; and wherein the antigen binding molecule selectively binds to OR8K3; wherein the cancer is a stomach cancer, esophageal cancer, ovarian cancer, renal cancer, or melanoma; and wherein the antigen binding molecule selectively binds to OR8B4; wherein the cancer is a stomach cancer, esophageal cancer, renal cancer, prostate cancer, or head and neck cancer; and wherein the antigen binding molecule selectively binds to OR6Q1; wherein the cancer is a renal cancer, stomach cancer, lung cancer, head and neck cancer, or melanoma; and wherein the antigen binding molecule selectively binds to OR5M8. For example, in one aspect, disclosed herein are methods of treating, reducing, inhibiting, decreasing, ameliorating and/or preventing a cancer and/or metastasis in a subject comprising administering to the subject one or more antigen binding molecules that selective binds to an OR5V1 olfactory receptor; and wherein the antigen binding molecule comprises one or more (for example, one, two, or three) complementarity determining regions (CDRs) as set forth in SEQ ID NO: 9, SEQ ID NO: 10, and SEQ ID NO: 11 (including, but not limited to an antigen binding molecule comprising the sequence as set forth in SEQ ID NO: 8); SEQ ID NO: 14, SEQ ID NO: 15, and SEQ ID NO: 16 (including, but not limited to an antigen binding molecule comprising the sequence as set forth in SEQ ID NO: 13); SEQ ID NO: 21, SEQ ID NO: 22, and SEQ ID NO: 23 (including, but not limited to an antigen binding molecule comprising the sequence as set forth in SEQ ID NO: 20); or SEQ ID NO: 26, SEQ ID NO: 27, and SEQ ID NO: 28 (including, but not limited to an antigen binding molecule comprising the sequence as set forth in SEQ ID NO: 25). Alternatively, for example, disclosed herein are methods of treating, reducing, inhibiting, decreasing, ameliorating and/or preventing a cancer and/or metastasis in a subject comprising administering to the subject one or more antigen binding molecules that selective binds to an OR2H1 olfactory receptor; and wherein the antigen binding molecule comprises one or more (for example, one, two, or three) complementarity determining regions (CDRs) as set forth in SEQ ID NO: 31, SEQ ID NO: 32, SEQ ID NO: 33 (including, but not limited to an antigen binding molecule comprising the sequence as set forth in SEQ ID NO: 34); SEQ ID NO: 35, SEQ ID NO: 36, and SEQ ID NO: 37 (including, but not limited to an antigen binding molecule comprising the sequence as set forth in SEQ ID NO: 38); SEQ ID NO: 41, SEQ ID NO: 42, and SEQ ID NO: 43 (including, but not limited to an antigen binding molecule comprising the sequence as set forth in SEQ ID NO: 40); SEQ ID NO: 45, SEQ ID NO: 46, and SEQ ID NO: 47 (including, but not limited to an antigen binding molecule comprising the sequence as set forth in SEQ ID NO: 44); SEQ ID NO: 53, SEQ ID NO: 54, and SEQ ID NO: 55 (including, but not limited to an antigen binding molecule comprising the sequence as set forth in SEQ ID NO: 48); and/or SEQ ID NO: 61, SEQ ID NO: 62, and SEQ ID NO: 63 (including, but not limited to an antigen binding molecule comprising the sequence as set forth in SEQ ID NO: 56). In one aspect the antigen binding molecule can comprise a variable heavy (V_{H}) chain sequence as set forth in SEQ ID NO: 65 and/or comprise a variable light (V_{L}) chain sequence as set forth in SEQ ID NO: 65.

In one aspect, the antigen binding molecule comprises a chimeric antigen receptor (such as, for example, a CAR T cell comprising a chimeric antigen receptor comprising the sequence as set forth in SEQ ID NO: 17). Also disclosed herein are methods of treating, reducing, inhibiting, decreasing, ameliorating and/or preventing a cancer and/or metastasis in a subject comprising administering to the subject one or more antigen binding molecules that selective binds to an OR2H1 olfactory receptor; and wherein the antigen binding molecule comprises one or more (for example, one, two, or three) variable heavy chain complementarity determining regions (CDRs) as set forth in SEQ ID NO: 31, SEQ ID NO: 32, and SEQ ID NO: 33 (including, but not limited to an antigen binding molecule comprising the variable heavy chain sequence as set forth in SEQ ID NO: 34); and/or variable light chain CDRs as set forth in SEQ ID NO: 35, SEQ ID NO: 36, and SEQ ID NO: 37 (including, but not limited to an antigen binding molecule comprising the variable light chain sequence as set forth in SEQ ID NO: 38). In one aspect, the antigen binding molecule comprises a chimeric antigen receptor (such as, for example, a CAR T cell comprising a chimeric antigen receptor comprising the sequence as set forth in SEQ ID NO: 30 or SEQ ID NO: 64).

Also disclosed herein are methods of treating, reducing, inhibiting, decreasing, ameliorating and/or preventing a cancer and/or metastasis, further comprising obtaining a tissue sample of a suspected cancerous tissue from the subject and assaying for the presence or overexpression of an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and/or OR5V1; wherein the presence of the olfactory receptor in the tissue sample not present in a negative control tissue sample or overexpression of the olfactory receptor in the tissue sample relative to a negative control tissue sample indicates the presence of a cancer in the subject.

In some aspect, it is understood and herein contemplated that while the expression of an olfactory receptor my occur in normal tissue, expression in a limited tissue set can be precisely why the olfactory receptor makes a good therapeutic target. This is particularly true where said expression is limited to an nonvital organ or tissue (such as, for example testicular tissue, prostatic tissue, ovarian tissue, breast tissue) and thus off-target issues are not only limited due to limited expression of the target, but also any noncancerous tissue destroyed by the therapeutic will not pose a life threatening effect on the treated subject. Thus, in one aspect, disclosed herein are methods of treating, reducing, inhibiting, decreasing, ameliorating and/or preventing a cancer and/or metastasis in a subject comprising administering to a subject with a cancer, an anticancer agent or binding molecule of any preceding aspect, wherein the cancer is present in a tissue that normally expresses the olfactory receptor when cancer is not present.

In one aspect, it is understood and herein contemplated that successful treatment of a cancer in a subject is important and doing so may include the administration of additional treatments. Thus, the disclosed methods of detecting/diagnosing and methods of treating, reducing, inhibiting, decreasing, ameliorating and/or preventing a cancer and/or metastasis can include or further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Where an EGFR splice variant isoform is not detected, the treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

E. Sequences

```
SEQ ID NO: 1 amino acid sequence of OR2H1 antigen
MVNQSSPMGFLLLGFSEHPALERTLFVVVFTSYLLTLVGNTLIILLSVLYPRLHSPMYFFL
SDLSFLDLCFTTSCVPQMLVNLWGPKKTISFLGCSVQLFIFLSLGTTECILLTVMAFDRYV

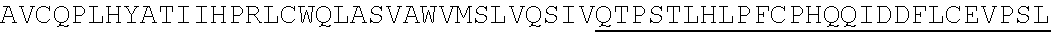

IRLSCGDTSYNEIQLAVSSVIFVVVPLSLILASYGATAQAVLRINSATAWRKAFGTCSSHL
TVVTLFYSSVIAVYLQPKNPYAQGRGKFFGLFYAVGTPSLNPLVYTLRNKEIKRALRRL
LGKERDSRESWRAA
SEQ ID NO: 2 amino acid sequence of OR52R1.
MVLASGNSSSHPVSFILLGIPGLESFQLWIAFPFCATYAVAVVGNITLLHVIRIDHTLHEP
MYLFLAMLAITDLVLSSSTQPKMLAIFWFHAHEIQYHACLIQVFFIHAFSSVESGVLMA
MALDCYVATCFPLRHSSILTPSVVIKLGTIVMLRGLLWVSPFCFMVSRMPFCQHQAIPQS
YCEHMAVLKLVCADTSISRGYGLFVAFSVAGFDMIVIGMSYVMILRAVLQLPSGEARLK
AFSTRASHICVILALYIPALFSFLTYRFGHDVPRVVHILFANLYLLIPPMLNPIIYGVRTKQI
GDRVIQGCCGNIP
SEQ ID NO: 3 amino acid sequence of OR2G3
MGLGNESSLMDFILLGFSDHPRLEAVLFVFVLFFYLLTLVGNFTIIIISYLDPPLHTPMYFF
LSNLSLLDICFTTSLAPQTLVNLQRPKKTITYGGCVAQLYISLALGSTECILLADMALDRY
IAVCKPLHYVVIMNPRLCQQLASISWLSGLASSLIHATFTLQLPLCGNHRLDHFICEVPAL
LKLACVDTTVNELVLFVVSVLFVVIPPALISISYGFITQAVLRIKSVEARHKAFSTCSSHLT
VVIIFYGTIIYVYLQPSDSYAQDQGKFISLFYTMVTPTLNPIIYTLRNKDMKEALRKLLSG
KL
SEQ ID NO: 4 amino acid sequence for OR5V1
MERKNQTAITEFIILGFSNLNELQFLLFTIFFLTYFCTLGGNILIILTTVTDPHLHTPMYYFL
GNLAFIDICYTTSNVPQMMVHLLSKKKSISYVGCVVQLFAFVFFVGSECLLLAAMAYDR
YIAICNPLRYSVILSKVLCNQLAASCWAAGFLNSVVHTVLTFCLPFCGNNQINYFFCDIPP
LLILSCGNTSVNELALLSTGVFIGWTPFLCIVLSYICIISTILRIQSSEGRRKAFSTCASHLAI
VFLFYGSAIFTYVRPISTYSLKKDRLVSVLYSVVTPMLNPIIYTLRNKDIKEAVKTIGSKW
QPPISSLDSKLTY
SEQ ID NO: 5 amino acid sequence for OR5V1 extracellular domain antigen

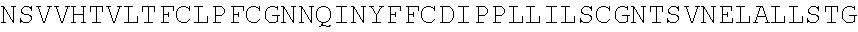

SEQ ID NO: 6 OR5V1 peptide for target sequence for antibody generation
TVLTFCLPFCGNNQINYF
SEQ ID NO: 7 nucleic acid sequence for IGHV1-18, IGHJ5, IGHM:
ATGGACTGGACCTGGAGCATCCTTTTCTTGGTGGCAGCAGCAACAGGTGCCCACTCC
CAGGTTCAGCTGCTGCAGTCTGGAGCTGAGGTGAAGAAGCCTGGGGCCTCAGTGAA
GGTCTCCTGCAAGGCTTCTGGTTACACCTTTACCAGCTATGGTATCACCTGGGTGCG
ACAGGCCCCTGGACAAGGGCTTGAGTGGATGGGATGGATCAGCGCTTACAATGGTA
ACACAAACTATGCACAGAAGCTCCAGGGCAGAGTCACCATGACCACAGACACATCC
ACGAGCACAGCCTACATGGACCTGAGGAGCCTGAGATCTGACGACACGGCCGTGTA
TTACTGTGCGAGAGCATCCGTGCCGGCCCCTATAACTGGAACTATCATCTGGTTCGA
CCCCTGGGGCCAGGGGACGCTGGTCACCGTCTCCTCAGGGAGTGCATCCGCCCCAA
CCCTTTTCCCCCTCGTCTCCTGTGAGAATTCCCCGTCGGATACGAGCAGCGTG
SEQ ID NO: 8 amino acid sequence for IGHV1-18, IGHJ5, IGHM:
        M D W T W S I L F L V A A A T G A H S Q V Q L L Q S G A E V
    1 ATGGACTGGACCTGGAGCATCCTTTTCTTGGTGGCAGCAGCAACAGGTGCCCACTCCCAGGTTCAGCTGCTGCAGTCTGGAGCTGAGGTG
        K K P G A S V K V S C K A S [symbol]   G I T W V R Q A P
  91 AAGAAGCCTGGGGCCTCAGTGAAGGTCTCCTGCAAGGCTTCTGGTTACACCTTTACCAGCTATGGTATCACCTGGGTGCGACAGGCCCCT
        G Q G L E W M G W I [symbol]   T N Y A Q K L Q G R V T M T
181 GGACAAGGGCTTGAGTGGATGGGATGGATCAGCGCTTACAATGGTAACACAAACTATGCACAGAAGCTCCAGGGCAGAGTCACCATGACC
        T D T S T S T A Y M D L R S L R S D D T A V Y Y C A R [symbol]
271 ACAGACACATCCACGAGCACAGCCTACATGGACCTGAGGAGCCTGAGATCTGACGACACGGCCGTGTATTACTGTGCGAGAGCATCCGTG

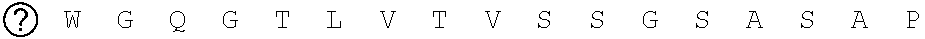

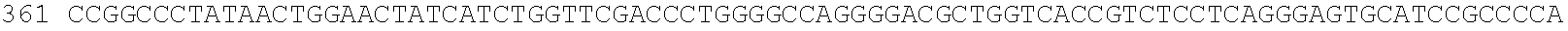

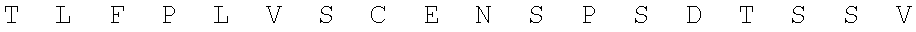

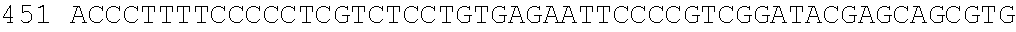




SEQ ID NO: 9 amino acid sequence for IGHV1-18, IGHJ5, IGHM CDR1
GYTFTSY
SEQ ID NO: 10 amino acid sequence for IGHV1-18, IGHJ5, IGHM CDR2
SAYNGN
SEQ ID NO: 11 amino acid sequence for IGHV1-18, IGHJ5, IGHM CDR3
ASVPAPITGTIIWFDP
SEQ ID NO: 12 nucleic acid sequence for IGLV3-21,IGLJ2, IGLC2:
ATGGCCTGGACCGTTCTCCTCCTCGGCCTCCTCTCTCACTGCACAGGCTCTGTGACCT
CCTACGTGCTGACTCAGCCACCCTCGGTGTCAGTGGCCCCAGGACAGACGGCCAGG
ATTACCTGTGGGGGAAACAACATTGGAAGTAAAAGTGTACACTGGTACCAGCAGAA
GCCAGGCCAGGCCCCTGTGCTGGTCGTCTATGATGATAGCGACCGGCCCTCAGGGA
TCCCTGAGCGATTCTCTGGCTCCAACTCTGGGAACACGGCCACCCTGACCATCAGCA
GGGTCGAAGCCGGGGATGAGGCCGACTATTACTGTCAGGTGTGGGATAGTAGTAGT
GATCATGTGGTATTCGGCGGAGGGACCAAGCTGACCGTCCTAGGTCAGCCCAAGGC
TGCCCCCTCGGTCACTCTGTTCCCGCCCTCCTCTGAGGAGCTTCAAGCCAACAAGGC
CACACTGGTGTGTCTCATAAGTGACTTCTACCCGGGAGCCGTGACAGTGGCCTGGA
AGGCAGATAGCAGCCCCGTCAAGGCGGGAGTGGAGACCACCACACCCTCCAAACA
AAGCAACAACAAGTACGCGGCCAGCAGCTA
SEQ ID NO: 13 amino acid sequence for IGLV3-21,IGLJ2, IGLC2
    1 ATGGCCTGGACCGTTCTCCTCCTCGGCCTCCTCTCTCACTGCACAGGCTCTGTGACCTCCTACGTGCTGACTCAGCCACCCTCGGTGTCA
        V A P G Q T A R I T C [symbol]   W Y Q Q K P G Q
  91 GTGGCCCCAGGACAGACGGCCAGGATTACCTGTGGGGGAAACAACATTGGAAGTAAAAGTGTACACTGGTACCAGCAGAAGCCAGGCCAG
        A P V L V V Y [symbol]   G I P E R F S G S N S G N T A T
181 GCCCCTGTGCTGGTCGTCTATGATGATAGCGACCGGCCCTCAGGGATCCCTGAGCGATTCTCTGGCTCCAACTCTGGGAACACGGCCACC
        L T I S R V E A G D E A D Y Y C [symbol]   F G G
271 CTGACCATCAGCAGGGTCGAAGCCGGGGATGAGGCCGACTATTACTGTCAGGTGTGGGATAGTAGTAGTGATCATGTGGTATTCGGCGGA

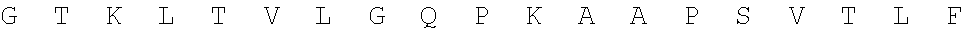

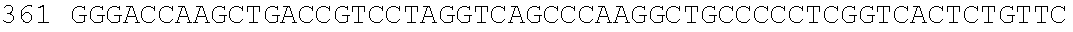


SEQ ID NO: 14 amino acid sequence for IGLV3-21,IGLJ2, IGLC2 CDR1
GGNNIGSKSVH
SEQ ID NO: 15 amino acid sequence for IGLV3-21,IGLJ2, IGLC2 CDR2
DDSDRPS
SEQ ID NO: 16 amino acid sequence for IGLV3-21,IGLJ2, IGLC2 CDR3
QVWDSSSDHVV
SEQ ID NO: 17 amino acid sequence Chimeric antigen receptor against OR5V1:
Signal peptide; VA; Spacer; VE; CD8 hinge domain; CD8 TM domain; 4-1BB IC
domain; CD3zeta
      1

[symbol]

[symbol]

    61 GTTCAGCTGCTGCAGTCTGGAGCTGAGGTGAAGAAGCCTGGGGCCTCAGTGAAGGTCTCC
        C K A [symbol]
  121 TGCAAGGCTTCTGGTTACACCTTTACCAGCTATGGTATCACCTGGGTGCGACAGGCCCCT

[symbol]

  181 GGACAAGGGCTTGAGTGGATGGGATGGATCAGCGCTTACAATGGTAACACAAACTATGCA

[symbol]

  241 CAGAAGCTCCAGGGCAGAGTCACCATGACCACAGACACATCCACGAGCACAGCCTACATG

[symbol]

  301 GACCTGAGGAGCCTGAGATCTGACGACACGGCCGTGTATTACTGTGCGAGAGCATCCGTG

[symbol]

  361 CCGGCCCCTATAACTGGAACTATCATCTGGTTCGACCCCTGGGGCCAGGGGACGCTGGTC [symbol]
          G G G G S G G G G S G G G G S [symbol]
  421 ACCGTCTCCTCAGGTGGAGGCGGTTCAGGCGGCGGTGGCTCTGGCGGTGGCGGATCGGGC

[symbol]

  481 TCTGTGACCTCCTACGTGCTGACTCAGCCACCCTCGGTGTCAGTGGCCCCAGGACAGACG

[symbol]

  541 GCCAGGATTACCTGTGGGGGAAACAACATTGGAAGTAAAAGTGTACACTGGTACCAGCAG

[symbol]

  601 AAGCCAGGCCAGGCCCCTGTGCTGGTCGTCTATGATGATAGCGACCGGCCCTCAGGGATC

[symbol]

  661 CCTGAGCGATTCTCTGGCTCCAACTCTGGGAACACGGCCACCCTGACCATCAGCAGGGTC

[symbol]

  721 GAAGTGGTATTCGGCGGAGGGACCAAGCTGACCGTCCTAAAGGIGGAAAACCACACCGCC

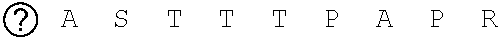

  781 TGCCACTGCAGCACATGCTACTACCACAAGAGCGCTAGCACCACCACCCCTGCCCCTAGA

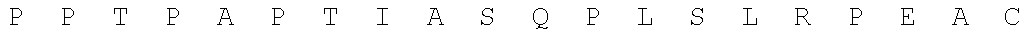

  841 CCTCCAACACCCGCCCCTACAATCGCCTCCCAGCCTCTGTCTCTGAGGCCCGAGGCTTGT

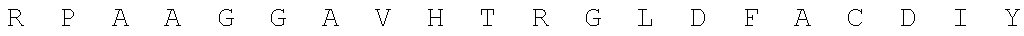

  901 AGACCAGCTGCTGGCGGAGCCGTGCACACCAGAGGACTGGATTTCGCCTGCGACATCTAC

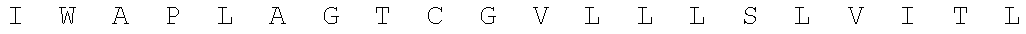

  961 ATCTGGGCCCCTCTGGCCGGCACATGTGGCGTGCTGCTGCTGAGCCTCGTGATCACCCTG

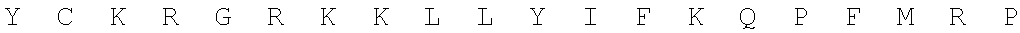

1021 TACTGCAAGCGGGGCAGAAAGAAGCTGCTGTACATCTTCAAGCAGCCCTTCATGCGGCCC
        V Q T T Q E E D G C S C R E P E E E E G
1081 GTGCAGACCACCCAGGAAGAGGACGGCTGCTCCTGCAGATTCCCCGAAGAGGAAGAGGGG

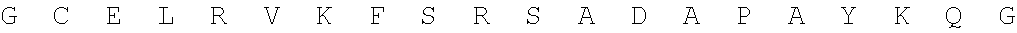

1141 GGCTGCGAACTGAGAGTGAAGTTCAGCAGAAGCGCCGACGCCCCTGCCTACAAGCAGGGC

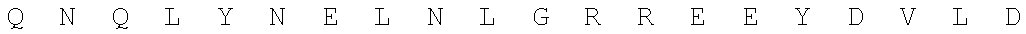

1201 CAGAACCAGCTGTACAACGAGCTGAACCTGGGCAGACGGGAAGAGTACGACGTGCTGGAC

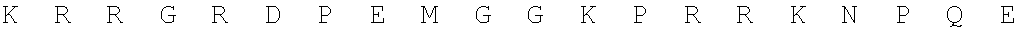

1261 AAGCGGAGAGGCAGGGACCCTGAGATGGGCGGAAAGCCCAGACGGAAGAACCCCCAGGAA

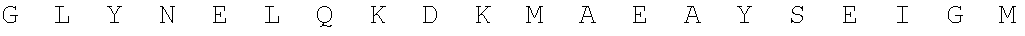

1321 GGCCTGTATAACGAACTGCAGAAAGACAAGATGGCCGAGGCCTACAGCGAGATCGGAATG

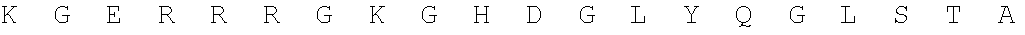

1381 AAGGGCGAGCGGAGAAGAGGCAAGGGCCACGATGGCCTGTACCAGGGCCTGAGCACCGCC

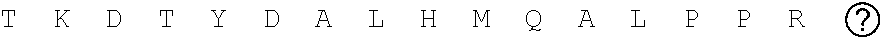

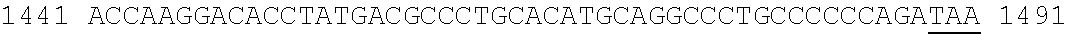


SEQ ID NO: 18 DNA sequence Chimeric antigen receptor against OR5V1:
ATGGACTGGACCTGGAGCATCCTTTTCTTGGTGGCAGCAGCAACAGGTGCCCACTCC
CAGGTTCAGCTGCTGCAGTCTGGAGCTGAGGTGAAGAAGCCTGGGGCCTCAGTGAA
GGTCTCCTGCAAGGCTTCTGGTTACACCTTTACCAGCTATGGTATCACCTGGGTGCG
ACAGGCCCCTGGACAAGGGCTTGAGTGGATGGGATGGATCAGCGCTTACAATGGTA
ACACAAACTATGCACAGAAGCTCCAGGGCAGAGTCACCATGACCACAGACACATCC
ACGAGCACAGCCTACATGGACCTGAGGAGCCTGAGATCTGACGACACGGCCGTGTA
TTACTGTGCGAGAGCATCCGTGCCGGCCCCTATAACTGGAACTATCATCTGGTTCGA
CCCCTGGGGCCAGGGGACGCTGGTCACCGTCTCCTCAGGTGGAGGCGGTTCAGGCG
GCGGTGGCTCTGGCGGTGGCGGATCGGGCTCTGTGACCTCCTACGTGCTGACTCAGC
CACCCTCGGTGTCAGTGGCCCCAGGACAGACGGCCAGGATTACCTGTGGGGGAAACA
ACATTGGAAGTAAAAGTGTACACTGGTACCAGCAGAAGCCAGGCCAGGCCCCTGTGCTG
GTCGTCTATGATGATAGCGACCGGCCCTCAGGGATCCCTGAGCGATTCTCTGGCTCCAAC
TCTGGGAACACGGCCACCCTGACCATCAGCAGGGTCGAAGTGGTATTCGGCGGAGGGAC
CAAGCTGACCGTCCTAAAGGTGGAAAACCACACCGCCTGCCACTGCAGCACATGCTACTA
CCACAAGAGCGCTAGCACCACCACCCCTGCCCCTAGACCTCCAACACCCGCCCCTACAA
TCGCCTCCCAGCCTCTGTCTCTGAGGCCCGAGGCTTGTAGACCAGCTGCTGGCGGAGCC
GTGCACACCAGAGGACTGGATTTCGCCTGCGACATCTACATCTGGGCCCCTCTGGCCGG
CACATGTGGCGTGCTGCTGCTGAGCCTCGTGATCACCCTGTACTGCAAGCGGGGCAGAA
AGAAGCTGCTGTACATCTTCAAGCAGCCCTTCATGCGGCCCGTGCAGACCACCCAGGAA
GAGGACGGCTGCTCCTGCAGATTCCCCGAAGAGGAAGAGGGGGGCTGCGAACTGAGAG
TGAAGTTCAGCAGAAGCGCCGACGCCCCTGCCTACAAGCAGGGCCAGAACCAGCTGTAC
AACGAGCTGAACCTGGGCAGACGGGAAGAGTACGACGTGCTGGACAAGCGGAGAGGCA
GGGACCCTGAGATGGGCGGAAAGCCCAGACGGAAGAACCCCCAGGAAGGCCTGTATAA
CGAACTGCAGAAAGACAAGATGGCCGAGGCCTACAGCGAGATCGGAATGAAGGGCGAGC
GGAGAAGAGGCAAGGGCCACGATGGCCTGTACCAGGGCCTGAGCACCGCCACCAAGGA
CACCTATGACGCCCTGCACATGCAGGCCCTGCCCCCCAGATAA
SEQ ID NO: 19 nucleic acid sequence for IGHV3-21, IGHJ6,IGHG1:
ATGGAACTGGGGCTCCGCTGGGTTTTCCTTGTTGCTATTTTAGAAGGTGTCCAGTGT
GAGGTGCAGCTGGTGGAGTCTGGGGGAGGCCTGGTCAAGCCTGGGGGGTCCCTGAG
ACTCTCCTGTGCAGCCTCTGGATTCACCTTCAGTAGCTATAGCATGACCTGGGTCCG
CCAGGCTCCAGGGAAGGGGCTGGACTGGGTCTCATCCATTAGTAGTAGTAGTAGTT
ACATATACTACGCAGACTCATTGAAGGGCCGATTCACCATCTCCAGAGACAACGCC
AAGAACTCACTGTATCTACAAATTAACAGCCTGAGAGTCGAGGACACGGCTGTGTA
TTACTGTGCGAGAGATCCTTCATTATGGCCGAAACCCCACTACTACTACGACGGTAT
GGACGTCTGGGGCCAAGGGACCACGGTCACCGTCTCCTCAGCCTCCACCAAGGGCC
CATCGGTCTTCCCCCTGGCACCCTCCTCCAAGAGCACCTCTGGGGGCACAGCGGCCC
TGGGCTGCCTGGTCAAGGACTACTTCCCCGAACCGGTGACGGTGTCGTGGAACTCA
GGCGCCCTGACCAGCGGCGTGCACACCTTCCCGGCTGTCCTACAGTCCTCAGGA
SEQ ID NO: 20 amino acid sequence for IGHV3-21, IGHJ6, IGHG1 [symbol]
        C E
    1 ATGGAACTGGGGCTCCGCTGGGTTITCCTTGTTGCTATITTAGAAGGTGTCCAGTGTGAG
        V Q L V E S G G G L V K P G G S L R L S
121 TGTGCAGCCTCTGGATTCACCTTCAGTAGCTATAGCATGACCTGGGTCCGCCAGGCTCCA
        G K G L D W V S S I [symbol]   Y I Y Y A
181 GGGAAGGGGCTGGACTGGGTCTCATCCATTAGTAGTAGTAGTAGTTACATATACTACGCA
        D S L K G R E T I S R D N A K N S L Y L
241 GACTCATTGAAGGGCCGATTCACCATCTCCAGAGACAACGCCAAGAACTCACTGTATCTA
        Q I N S L R V E D T A V Y Y C A R D P S
301 CAAATTAACAGCCTGAGAGTCGAGGACACGGCTGTGTATTACTGTGCGAGAGATCCTTCA [symbol]
          M D V W G Q G T T
361 TTATGGCCGAAACCCCACTACTACTACGACGGTATGGACGTCTGGGGCCAAGGGACCACG
        V T V S S
421 GTCACCGTCICCICA 435
SEQ ID NO: 21 amino acid sequence for IGHV3-21, IGHJ6, IGHG1 CDR1
GFTFSSY
SEQ ID NO: 22 amino acid sequence for IGHV3-21, IGHJ6, IGHG1 CDR2
SSSSSY
SEQ ID NO: 23 amino acid sequence for IGHV3-21, IGHJ6, IGHG1 CDR3
DPSLWPKPHYYYDGM
SEQ ID NO: 24 nucleic acid sequence for IGKV3-20,IGKJ5,IGKC:
ATGGAAACCCCAGCGCAGCTTCTCTTCCTCCTGCTACTCTGGCTCCCAGATACCACC
GGAGAAATTGTGTTGACGCAGTCTCCAGGCACCCTGTCTTTGTCTCCAGGGGAAAG
AGCCACCCTCTCCTGCAGGGCCAGTCAGAGTGTTAGCCGCAGCTATTTAGCCTGGTA
CCAGCAGAAACCTGGCCAGGCTCCCAGGCTCCTCATCTATGGTGCATCCAGCAGGG
CCACTGGCATCCCAGACAGGTTCAGTGGCAGTGAGTCTGGGACAGACTTCACTCTC
ACCATCAACAGACTGGAGCCTGAAGATTTTGCACTGTATTACTGTCAGCTATATGGT
AACTCTCTGATCACCTTCGGCCAAGGGACACGACTGGAGATTAAACGAACTGTGGC
TGCACCATCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTACAGTGGAA
GGTGGATAACGC
SEQ ID NO: 25 amino acid sequence for IGKV3-20, IGKJ5, IGKC [symbol]
          G
    1 ATGGAAACCCCAGCGCAGCTTCTCTTCCTCCTGCTACTCTGGCTCCCAGATACCACCGGA
        E I V L T Q S P G T L S L S P G E R A T
  61 GAAATTGTGTIGACGCAGTCTCCAGGCACCCTGTCTTTGTCTCCAGGGGAAAGAGCCACC
        L S C P [symbol]   W Y Q Q K
121 CTCTCCTGCAGGGCCAGTCAGAGTGTTAGCCGCAGCTATTTAGCCTGGTACCAGCAGAAA
        P G Q A P R L L I Y [symbol]   G I P
181 CCTGGCCAGGCTCCCAGGCTCCTCATCTATGGTGCATCCAGCAGGGCCACIGGCATCCCA
        D R E S G S E S G T D E T L T I N R L E
241 GACAGGTTCAGTGGCAGTGAGTCTGGGACAGACTTCACTCTCACCATCAACAGACTGGAG
        P E D F A L Y Y C [symbol]   F G
301 CCTGAAGATTITGCACTGTATTACTGTCAGCTATATGGTAACTCTCTGATCACCTTCGGC
        Q G T R L E I K
361 CAAGGGACACGACTGGAGATTAAA 384
SEQ ID NO: 26 amino acid sequence for IGKV3-20,IGKJ5, IGKC CDR1
RASQSVSRSYLA
SEQ ID NO: 27 amino acid sequence for IGKV3-20,IGKJ5, IGKC CDR2
GASSRAT
SEQ ID NO: 28 amino acid sequence for IGKV3-20,IGKJ5, IGKC CDR3
QLYGNSLIT
SEQ ID NO: 29 nucleic acid sequence for pBMN-GFP

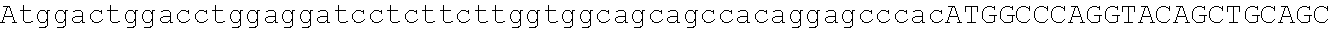

AGTCAGGAGCAGAGGTGAAAAAGCCGGGGGAATCTCTGAAGATCTCCTGTAAGGGT
TCTGGATACATCTTTACCGGCTACTATATGCACTGGGTGCGACAGGCCCCTGGACAA
CGGCCTGAGTGGCTGGGACGGATGAACGCAAAAAGTGGTGAGGCAGACTCTGCAC
AGAGATTTCAGGGCAGGGTCACCATGACCAGAGACACGTCCATCAATACAGCCTAC
ATGGAACTGAGAGACCTGAGATCTGACGACACGGCCGTGTATTATTGTACGAGAGG
TCCCCTGCTCTGGGGCCAGGGAACCCTGGTCACCGTCTCGAGTGGTGGTGGCGGTTC
TGGTGGTGGTGGTAGCGGTGGCGGTGGTAGTGGCGGTGGCGGTGCTAGCCAGCCTG
TGCTGACTCAGCCACCCTCAGCGTCTGGGACCCCCGGGCAGAGGGTCACCATCTCCT
GTTCTGGAAGCAGCTCCAACATCGGGAGTAATTTTGTATCCTGGTACCAGCAGCTCC
CAGGAACGGCCCCCAAACTCCTCATCTATAGGAATAATCAGCGGCCCTCAGGGGTC
CCTGACCGATTCTCTGGCTCCAAGACTGGCACCTCAGCCTCCCTGGCCATCAGTGGG
CTCCGGTCCGAGGATGAGGCTGATTATTACTGTGCAGCATGGGATGACAGTGTGAG
AGGTCCGGTGTTCGGCGGAGGGACCGAGCTGACCGTCCTAGCGGCCGCAACCACCA
CCCCTGCCCCTAGACCTCCAACACCCGCCCCTACAATCGCCTCCCAGCCTCTGTCTCTGA
GGCCCGAGGCTTGTAGACCAGCTGCTGGCGGAGCCGTGCACACCAGAGGACTGGATTTC
GCCTGCGACATCTACATCTGGGCCCCTCTGGCCGGCACATGTGGCGTGCTGCTGCTGAG
CCTCGTGATCACCCTGTACTGCAAGCGGGGCAGAAAGAAGCTGCTGTACATCTTCAAGCA
GCCCTTCATGCGGCCCGTGCAGACCACCCAGGAAGAGGACGGCTGCTCCTGCAGATTCC
CCGAAGAGGAAGAGGGGGGCTGCGAACTGAGAGTGAAGTTCAGCAGAAGCGCCGACGC
CCCTGCCTACAAGCAGGGCCAGAACCAGCTGTACAACGAGCTGAACCTGGGCAGACGGG
AAGAGTACGACGTGCTGGACAAGCGGAGAGGCAGGGACCCTGAGATGGGCGGAAAGCC
CAGACGGAAGAACCCCCAGGAAGGCCTGTATAACGAACTGCAGAAAGACAAGATGGCCG
AGGCCTACAGCGAGATCGGAATGAAGGGCGAGCGGAGAAGAGGCAAGGGCCACGATGG
CCTGTACCAGGGCCTGAGCACCGCCACCAAGGACACCTATGACGCCCTGCACATGCAGG
SEQ ID NO: 30 amino acid sequence for Chimeric antigen receptor against OR2H1 [symbol]
; VH; Spacer; VL; CD8 hinge domain; CD8 TM domain; 4-1BB IC
domain; CD3zeta CDR Region
      1 [symbol]   ATGGCC
    61 CAGGTACAGCTGCAGCAGTCAGGAGCAGAGGIGAAAAAGCCGGGGGAATCTCTGAAGATC

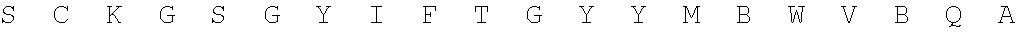

121 TCCTGIAAGGGITCIGGATACAICTITACCGGCIACIATATGCACIGGGTGCGACAGGCC
SGORBEWEGRM
  181 CCIGGACAACGGCCIGAGTGGCIGGGACGGAIGAACGCAAAAAGTGGTGAGGCAGACTCI

[symbol]

  241 GCACAGAGATTTCAGGGCAGGGTCACCATGACCAGAGACACGTCCATCAATACAGCCTAC

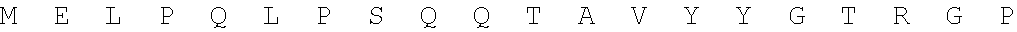

  301 ATGGAACTGAGAGACCTGAGATCTGACGACACGGCCGTGTATTATTGTACGAGAGGTCCC

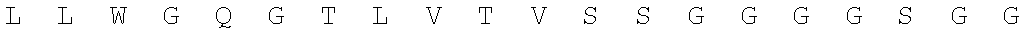

  361 CTGCTCTGGGGCCAGGGAACCCTGGTCACCGTCTCGAGTGGTGGTGGCGGTTCTGGTGGT
        G G S G G G G S G G G G A S [symbol]   Q
  421 GGTGGTAGCGGTGGCGGTGGTAGTGGCGGTGGCGGTGCTAGCCAGCCTGTGCTGACTCAG

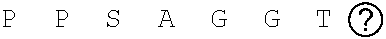

  481 CCACCCTCAGCGTCTGGGACCCCCGGGCAGAGGGTCACCATCTCCTGTTCTGGAAGCAGC

[symbol]

  541 TCCAACATCGGGAGTAATTTTGTATCCTGGTACCAGCAGCTCCCAGGAACGGCCCCCAAA

[symbol]

  601 CTCCTCATCTATAGGAATAATCAGCGGCCCTCAGGGGTCCCTGACCGATTCTCTGGCTCC
        K T G I S A S L A I S G L R S E D E A D
  661 AAGACTGGCACCTCAGCCTCCCTGGCCATCAGTGGGCTCCGGTCCGAGGATGAGGCTGAT

[symbol]

  721 TATTACTGTGCAGCATGGGATGACAGTGTGAGAGGTCCGGTGTTCGGCGGAGGGACCGAG

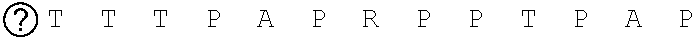

  781 CTGACCGTCCTAGCGGCCGCAACCACCACCCCTGCCCCTAGACCTCCAACACCCGCCCCT

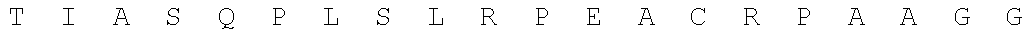

  841 ACAATCGCCTCCCAGCCTCTGICTCTGAGGCCCGAGGCTTGTAGACCAGCTGCTGGCGGA

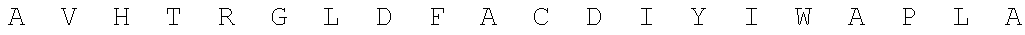

  961 GGCACATGTGGCGTGCTGCTGCTGAGCCTCGTGATCACCCTGTACTGCAAGCGGGGCAGA
        K K L L Y I F K Q P F M R P V Q T T Q E
1021 AAGAAGCTGCTGTACATCTTCAAGCAGCCCTTCATGCGGCCCGTGCAGACCACCCAGGAA
        E D G C S C R F P E E E E G G C E L R V
1081 GAGGACGGCTGCTCCTGCAGATTCCCCGAAGAGGAAGAGGGGGGCTGCGAACTGAGAGTG

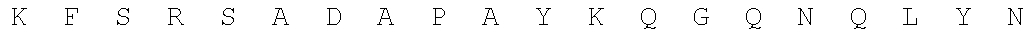

1141 AAGTTCAGCAGAAGCGCCGACGCCCCTGCCTACAAGCAGGGCCAGAACCAGCTGTACAAC

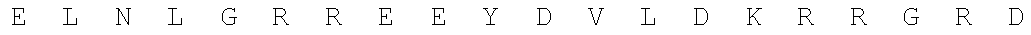

1201 GAGCTGAACCTGGGCAGACGGGAAGAGTACGACGTGCTGGACAAGCGGAGAGGCAGGGAC

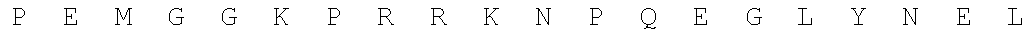

1261 CCTGAGATGGGGGGAAAGCCCAGACGGAAGAACCCCCAGGAAGGCCTGTATAACGAACTG

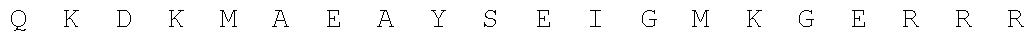

1321 CAGAAAGACAAGATGGCCGAGGCCTACAGCGAGATCGGAATGAAGGGCGAGCGGAGAAGA

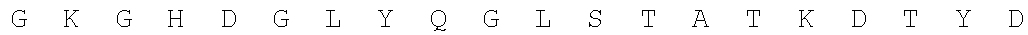

1381 GGCAAGGGCCACGATGGCCTGTACCAGGGCCTGAGCACCGCCACCAAGGACACCTATGAC

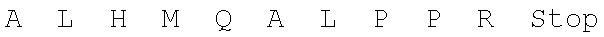

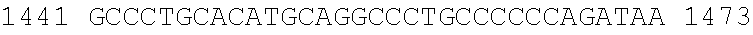


SEQ ID NO: 31 amino acid sequence for CDR 1 of V_{H} of Chimeric antigen receptor against
OR2H1
GYIFTGYY
SEQ ID NO: 32 amino acid sequence for CDR 2 of V_{H} of Chimeric antigen receptor against
OR2H1
NAKSGE
SEQ ID NO: 33 amino acid sequence for CDR 3 of V_{H} of Chimeric antigen receptor against
OR2H1
GPLL
SEQ ID NO: 34 amino acid sequence for Variable Heavy (V_{H}) chain of Chimeric antigen receptor
against OR2H1
QVQLQQSGAEVKKPGESLKISCKGSGYIFTGYYMHWVRQAPGQRPEWLGRMNAKSGE
ADSAQRFQGRVTMTRDTSINTAYMELRDLRSDDTAVYYCTRGPLLWGQGTLVTVSS
SEQ ID NO: 35 amino acid sequence for CDR 1 of V_{L} of Chimeric antigen receptor against
OR2H1
SSGSSNIGSNFVS
SEQ ID NO: 36 amino acid sequence for CDR 2 of V_{L} of Chimeric antigen receptor against
OR2H1
RNNQRPS
SEQ ID NO: 37 amino acid sequence for CDR 3 of V_{L} of Chimeric antigen receptor against
OR2H1
AAWDDSVRGPV
SEQ ID NO: 38 amino acid sequence for variable light (V_{L}) chain of Chimeric antigen receptor
against OR2H1
QPVLTQPPSASGTPGQRVTISCSGSSSNIGSNFVSWYQQLPGTAPKLLIYRNNQRPSGVPD
RFSGSKTGTSASLAISGLRSEDEADYYCAAWDDSVRGPVFGGGTELTVLAAA
SEQ ID NO: 39 amino acid sequence for ScFv against OR2H1 yellow linker; green VH
CDRs; blue VL CDRs Clone 1314-R3P1-E8
MAQVQLQQSGAEVKKPGESLKISCKGSGYIFTGYYMHWVRQAPGQRPEWLGRMNAKS
GEADSAQRFQGRVTMTRDTSINTAYMELRDLRSDDTAVYYCTRGPLLWGQGTLVTVSS
GGGGSGGGGSGGGGSGGGGASQPVLTQPPSASGTPGQRVTISCSGSSSNIGSNFVSWYQ
QLPGTAPKLLIYRNNQRPSGVPDRESGSKTGTSASLAISGLRSEDEADYYCAAWDDSVR
GPVFGGGTELTVLAAA
SEQ ID NO: 40 amino acid sequence for variable heavy (V_{H}) chain of ScFv against OR2H1
Clone 1314-R3P1-E8
MAQVQLQQSGAEVKKPGESLKISCKGSGYIFTGYYMHWVRQAPGQRPEWLGRMNAKS
GEADSAQRFQGRVTMTRDTSINTAYMELRDLRSDDTAVYYCTRGPLLWGQGTLVTVSS
SEQ ID NO: 41 amino acid sequence for variable heavy (V_{H}) chain CDR1 of ScFv against
OR2H1 Clone 1314-R3P1-E8
GYIFTGYY
SEQ ID NO: 42 amino acid sequence for variable heavy (V_{H}) chain CDR2 of ScFv against
OR2H1 Clone 1314-R3P1-E8
MNAKSGEA
SEQ ID NO: 43 amino acid sequence for variable heavy (V_{H}) chain CDR3 of ScFv against
OR2H1 Clone 1314-R3P1-E8
TRGPLL
SEQ ID NO: 44 amino acid sequence for variable light (V_{L}) chain of ScFv against OR2H1
Clone 1314-R3P1-E8
QPVLTQPPSASGTPGQRVTISCSGSSSNIGSNFVSWYQQLPGTAPKLLIYRNNQRPSGVPD
RFSGSKTGTSASLAISGLRSEDEADYYCAAWDDSVRGPVFGGGTELTVL
SEQ ID NO: 45 amino acid sequence for variable light (V_{L}) chain CDR1 of ScFv against
OR2H1 Clone 1314-R3P1-E8
SSNIGSNF
SEQ ID NO: 46 amino acid sequence for variable light (V_{L}) chain CDR2 of ScFv against
OR2H1 Clone 1314-R3P1-E8
RNN
SEQ ID NO: 47 amino acid sequence for variable light (V_{L}) chain CDR3 of ScFv against
OR2H1 Clone 1314-R3P1-E8
AAWDDSVRGPV
SEQ ID NO: 48 amino acid sequence for variable heavy (V_{H}) chain of ScFv against OR2H1
Clone 13134-R2P1-A1
EVQLVQSGAEVKKPGASVKVSCKASGYTFTSNYMHWVRQAPGQGLEWMGIINPSGGR
TSYAQKFQGRVTMTRDTSTGTVYMELSSLRSEDTAVYYCARSHCSGGSCYSIDYWGQG
TLVTVSS
SEQ ID NO: 49 amino acid sequence for FR1 of the variable heavy (V_{H}) chain of ScFv
against OR2H1 Clone 13134-R2P1-A1
EVQLVQSGAEVKKPGASVKVSCKAS
SEQ ID NO: 50 amino acid sequence for FR2 of the variable heavy (V_{H}) chain of ScFv
against OR2H1 Clone 13134-R2P1-A1
MHWVRQAPGQGLEWMGI
SEQ ID NO: 51 amino acid sequence for FR3 of the variable heavy (V_{H}) chain of ScFv
against OR2H1 Clone 13134-R2P1-A1
SYAQKFQGRVTMTRDTSTGTVYMELSSLRSEDTAVYYC
SEQ ID NO: 52 amino acid sequence for FR4 of the variable heavy (V_{H}) chain of ScFv
against OR2H1 Clone 13134-R2P1-A1
WGQGTLVTVSS
SEQ ID NO: 53 amino acid sequence for CDR1 of the variable heavy (V_{H}) chain of ScFv
against OR2H1 Clone 13134-R2P1-A1
GYTFTSNY
SEQ ID NO: 54 amino acid sequence for CDR2 of the variable heavy (V_{H}) chain of ScFv
against OR2H1 Clone 13134-R2P1-A1
INPSGGRT
SEQ ID NO: 55 amino acid sequence for CDR3 of the variable heavy (V_{H}) chain of ScFv
against OR2H1 Clone 13134-R2P1-A1
ARSHCSGGSCYSIDY
SEQ ID NO: 56 amino acid sequence for variable light (V_{L}) chain of ScFv against OR2H1
Clone 13134-R2P1-A1
QPVLTQSSSASASLGSSVKLTCTLSSGHSGYIIAWHQQQPGKAPRYLMKVEGSGSYNKG
SGIPERFSGSSSGADRYLTISNLQSEDEADYYCETWDSNTHVFGTGTKVTVL
SEQ ID NO: 57 amino acid sequence for FR1 of the variable light (V_{L}) chain of ScFv
against OR2H1 Clone 13134-R2P1-A1
QPVLTQSSSASASLGSSVKLTCTLS
SEQ ID NO: 58 amino acid sequence for FR2 of the variable light (V_{L}) chain of ScFv
against OR2H1 Clone 13134-R2P1-A1
IAWHQQQPGKAPRYLMK
SEQ ID NO: 59 amino acid sequence for FR3 of the variable light (V_{L}) chain of ScFv
against OR2H1 Clone 13134-R2P1-A1
NKGSGIPERFSGSSSGADRYLTISNLQSEDEADYYC
SEQ ID NO: 60 amino acid sequence for FR4 of the variable light (V_{L}) chain of ScFv
against OR2H1 Clone 13134-R2P1-A1
FGTGTKVTVL
SEQ ID NO: 61 amino acid sequence for CDR1 of the variable light (V_{L}) chain of ScFv
against OR2H1 Clone 13134-R2P1-A1
SGHSGYI
SEQ ID NO: 62 amino acid sequence for CDR2 of the variable light (V_{L}) chain of ScFv
against OR2H1 Clone 13134-R2P1-A1
VEGSGSY
SEQ ID NO: 63 amino acid sequence for CDR3 of the variable light (V_{L}) chain of ScFv
against OR2H1 Clone 13134-R2P1-A1
ETWDSNTHV
SEQ ID NO: 64 amino acid sequence OR2H1 CAR T cell TCR
Signal peptide; VA; Spacer; CD8 hinge domain; CD8 TM domain; 4-1BB IC
domain; CD3zeta

[symbol]

        1 [symbol]   tccgag
    61 gtgcagctggtgcagtetggggctgaggtgaagaagectggggcctcagtgaaggtttcc

[symbol]

  121 tgcaaggcatctggatacaccttcaccagcaactatatgcactgggtgcgacaggcecct

[symbol]

  181 ggacaagggcttgagtggatgggaataatcaaccctagtggtggtegcacaagotacgca

[symbol]

  241 cagaagttccagggcagagtcaccatgaccagggacacgtccacgagcacagtctacatg

[symbol]

  301 gagctgagcagoctgagatctgaggacacggocgtgtattactgtgcgagatcccattgc

[symbol]

  361 agcggcggcagctgctatagcattGACTACTGGGGCCAAGGAACCCTGGTCACCGTCTCC
        S G G G G S G G G G S G G G G S G G G G
  481 cagcctgtgctgactcaatcatectctgcctctgcttccctgggatcetcggtcaagctc
        T C T L S G G H G G Y T I A W M Q Q Q P
  541 acctgcactctgagcagtgggcacagtggctacatcatcgcatggcatcagcagcagcca

[symbol]

  601 gggaaggcccctcggtacttgatgaaggttgaaggtagtggaagotacaacaaggggagc
        G V P D P F S G S S S G A D R Y L T I S
  661 ggagttcctgatcgottctcaggctccagctctggggctgaccgetacctcaccatetcc

[symbol]

  721 aacctccagtttgaggatgaggctgattattactgtgagacctgggacagtaacactcaC

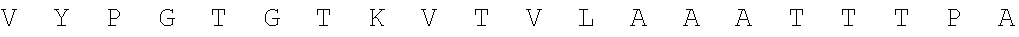

  781 GTGTActtoggaactgggaccaaggtcaccgtcotaGCGGCCGCAACCACCACCCCTGCC

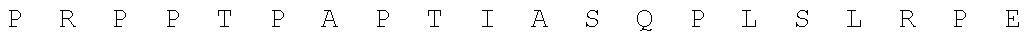

  841 CCTAGACCTCCAACACCCGCCCCTACAATCGCCTCCCAGCCTCTGTCTCTGAGGCCCGAG

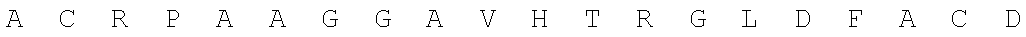

  901 GCTTGTAGACCAGCTGCTGGCGGAGCCGTGCACACCAGAGGACTGGATTTCGCCTGCGAC

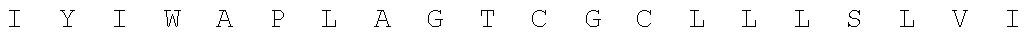

  961 ATCTACATCTGGGCCCCTCTGGCCGGCACATGTGGCGTGCTGCTGCTGAGCCTCGTGATC

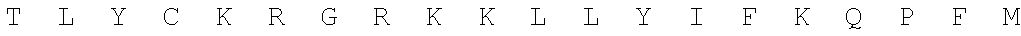

1021 ACCCTGTACTGCAAGCGGGGCAGAAAGAAGCTGCTGTACATCTTCAAGCAGCCCTTCATG
        R P V Q T T Q E E D G C S C R F P E E E
1081 CGGCCCGTGCAGACCACCCAGGAAGAGGACGGCTGCTCCTGCAGATTCCCCGAAGAGGAA

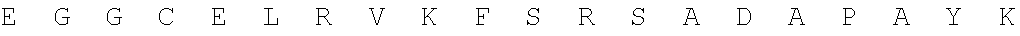

1141 GAGGGGGGCTGCGAACTGAGAGTGAAGTTCAGCAGAAGCGCCGACGCCCCIGCCTACAAG

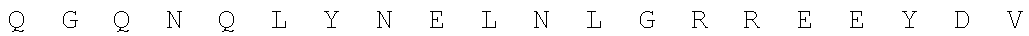

1201 CAGGGCCAGAACCAGCTGTACAACGAGCTGAACCTGGGCAGACGGGAAGAGTACGACGTG

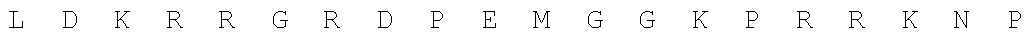

1261 CTGGACAAGCGGAGAGGCAGGGACCCTGAGATGGGCGGAAAGCCCAGACGGAAGAACCCC

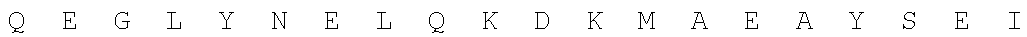

1321 CAGGAAGGCCTGTATAACGAACTGCAGAAAGACAAGATGGCCGAGGCCTACAGCGAGATC

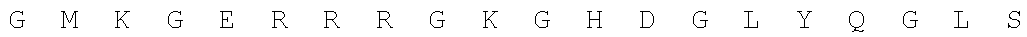

1381 GGAATGAAGGGCGAGCGGAGAAGAGGCAAGGGCCACGATGGCCTGTACCAGGGCCTGAGC

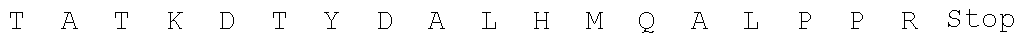

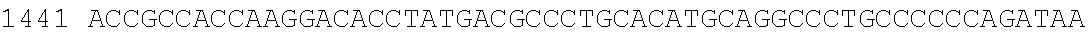


SEQ ID NO: 65 amino acid sequence for variable heavy (V_{H}) chain of OR2H1 CAR T cell
TCR
SEVQLVQSGAEVKKPGASVKVSCKASGYTFTSNYMHWVRQAPGQGLEWMGIINPSGG
RTSYAQKFQGRVTMTRDTSTSTVYMELSSLRSEDTAVYYCARSHCSGGSCYSIDYWGQ
GTLVTVSS
SEQ ID NO: 66 amino acid sequence for variable light (V_{L}) chain of OR2H1 CAR T cell
TCR
QPVLTQSSSASASLGSSVKLTCTLSSGHSGYIIAWHQQQPGKAPRYLMKVEGSGSYNKG
SGVPDRFSGSSSGADRYLTISNLQFEDEADYYCETWDSNTHVYFGTGTKVTVLAAA [symbol]
indicates data missing or illegible when filed
```

1. An antigen binding molecule that selectively binds to an olfactory receptor selected from the group consisting of OR2H1, OR52R1, OR56A3, OR12D2, OR2G3, OR8B2, OR8B3, OR51F1, OR8K3, OR8B4, OR6Q1, OR5M8, and OR5V1.

2. The antigen binding molecule of claim 1, wherein the antigen binding molecule comprises an RNAi, peptide, protein, chimeric antigen receptor (CAR) T cell, CAR NK cell, CAR macrophage (CARMA), siRNA, immunotoxin, diabody, antibody, or a functional antibody fragment.

3. The antigen binding molecule of claim 1 wherein the antigen binding molecule selectively binds to an olfactory receptor OR5V1 as set forth in SEQ ID NO: 4.

4. The antigen binding molecule of claim 1 wherein the antigen binding molecule selectively binds to the extracellular domain of an olfactory receptor OR5V1 as set forth in SEQ ID NO: 5.

5. The antigen binding molecule of claim 1 wherein the antigen binding molecule selectively binds to an olfactory receptor OR2H1 as set forth in SEQ ID NO: 1.

6. The antigen binding molecule of any of claims 1-5, wherein the antigen binding molecule comprises one or more complementarity determining regions (CDRs) as set forth in SEQ ID NO: 9, SEQ ID NO: 10, SEQ ID NO: 11, SEQ ID NO: 14, SEQ ID NO: 15, SEQ ID NO: 16, SEQ ID NO: 21, SEQ ID NO: 22, SEQ ID NO: 23, SEQ ID NO: 26, SEQ ID NO: 27, SEQ ID NO: 28; SEQ ID NO: 31, SEQ ID NO: 32, SEQ ID NO: 33, SEQ ID NO: 35, SEQ ID NO: 36, SEQ ID NO: 37, SEQ ID NO: 41, SEQ ID NO: 42, SEQ ID NO: 43, SEQ ID NO: 45, SEQ ID NO: 46, SEQ ID NO: 47, SEQ ID NO: 53, SEQ ID NO: 54, SEQ ID NO: 55, SEQ ID NO: 61, SEQ ID NO: 62, and/or SEQ ID NO: 63.

7. The antigen binding molecule of claim 6, wherein the antigen binding molecule selectively binds to an olfactory receptor OR5V1 and comprises one or more variable heavy (V_{H}) chain complementarity determining regions (CDRs) as set forth in SEQ ID NO: 9, SEQ ID NO: 10, and SEQ ID NO: 11.

8. The antigen binding molecule of claim 7, wherein the antigen binding molecule comprises the variable heavy (V_{H}) chain sequence as set forth in SEQ ID NO: 8

9. The antigen binding molecule of claim 6, wherein the antigen binding molecule selectively binds to an olfactory receptor OR5V1 and comprises one or more variable light (V_{L}) chain complementarity determining regions (CDRs) as set forth in SEQ ID NO: 14, SEQ ID NO: 15, and SEQ ID NO: 16.

10. The antigen binding molecule of claim 9, wherein the antigen binding molecule comprises the variable light (V_{L}) chain sequence as set forth in SEQ ID NO: 13.

11. The antigen binding molecule of claim 6, wherein the antigen binding molecule comprises one or more complementarity determining regions (CDRs) as set forth in SEQ ID NO: 21, SEQ ID NO: 22, and SEQ ID NO: 23.

12. The antigen binding molecule of claim 11, wherein the antigen binding molecule comprises the sequence as set forth in SEQ ID NO: 20.

13. The antigen binding molecule of claim 6, wherein the antigen binding molecule comprises one or more complementarity determining regions (CDRs) as set forth in SEQ ID NO: 26, SEQ ID NO: 27, and SEQ ID NO: 28.

14. The antigen binding molecule of claim 13, wherein the antigen binding molecule comprises the sequence as set forth in SEQ ID NO: 25.

15. The antigen binding molecule of claim 6, wherein the antigen binding molecule selectively binds to an olfactory receptor OR2H1 and comprises one or more variable heavy (V_{H}) chain complementarity determining regions (CDRs) as set forth in SEQ ID NO: 31, SEQ ID NO: 32, and SEQ ID NO: 33.

16. The antigen binding molecule of claim 15, wherein the antigen binding molecule comprises the variable heavy (V_{H}) chain sequence as set forth in SEQ ID NO: 34

17. The antigen binding molecule of claim 6, wherein the antigen binding molecule selectively binds to an olfactory receptor OR2H1 and comprises one or more variable light (V_{L}) chain complementarity determining regions (CDRs) as set forth in SEQ ID NO: 35, SEQ ID NO: 36, and SEQ ID NO: 37.

18. The antigen binding molecule of claim 17, wherein the antigen binding molecule comprises the variable light (V_{L}) chain sequence as set forth in SEQ ID NO: 38.

19. The antigen binding molecule of claim 6, wherein the antigen binding molecule selectively binds to an olfactory receptor OR2H1 and comprises one or more variable heavy (V_{H}) chain complementarity determining regions (CDRs) as set forth in SEQ ID NO: 41, SEQ ID NO: 42, and SEQ ID NO: 43.

20. The antigen binding molecule of claim 19, wherein the antigen binding molecule comprises the variable heavy (V_{H}) chain sequence as set forth in SEQ ID NO: 40

21. The antigen binding molecule of claim 6, wherein the antigen binding molecule selectively binds to an olfactory receptor OR2H1 and comprises one or more variable light (V_{L}) chain complementarity determining regions (CDRs) as set forth in SEQ ID NO: 45, SEQ ID NO: 46, and SEQ ID NO: 47.

22. The antigen binding molecule of claim 21, wherein the antigen binding molecule comprises the variable light (V_{L}) chain sequence as set forth in SEQ ID NO: 44.

23. The antigen binding molecule of claim 6, wherein the antigen binding molecule selectively binds to an olfactory receptor OR2H1 and comprises one or more variable heavy (V_{H}) chain complementarity determining regions (CDRs) as set forth in SEQ ID NO: 53, SEQ ID NO: 54, and SEQ ID NO: 55.

24. The antigen binding molecule of claim 23, wherein the antigen binding molecule comprises the variable heavy (V_{H}) chain sequence as set forth in SEQ ID NO: 48

25. The antigen binding molecule of claim 6, wherein the antigen binding molecule selectively binds to an olfactory receptor OR2H1 and comprises one or more variable light (V_{L}) chain complementarity determining regions (CDRs) as set forth in SEQ ID NO: 61, SEQ ID NO: 62, and SEQ ID NO: 63.

26. The antigen binding molecule of claim 25, wherein the antigen binding molecule comprises the variable light (V_{L}) chain sequence as set forth in SEQ ID NO: 56.

27. The antigen binding molecule of any of claims 1, 2, or 5, wherein the antigen binding molecule comprises the variable heavy (V_{H}) chain sequence as set forth in SEQ ID NO: 65

28. The antigen binding molecule of claim 1, wherein the antigen binding molecule comprises the variable light (V_{L}) chain sequence as set forth in SEQ ID NO: 66.

29. The antigen binding molecule of claim 2, wherein the antigen binding molecule comprises a chimeric antigen receptor (CAR) T cell.

30. The antigen binding molecule of any of claim 29, wherein CAR T cell comprises the sequence as set forth in SEQ ID NO: 17, SEQ ID NO: 30, or SEQ ID NO: 64.

31. A method of treating, inhibiting, reducing, decreasing, ameliorating, and/or preventing a cancer and/or metastasis in a subject comprising administering to the subject any of the antigen binding molecules of claim 1.

32. A method of detecting a cancer in a subject comprising obtaining a tissue sample of a suspected cancerous tissue from the subject and contacting the tissue sample with the antigen binding molecule of claim 1.

33-115. (canceled)


SEQUENCE LISTING (66 sequences)

SEQ ID NO: 1 - protein, 316 aa, Homo sapiens
MVNQSSPMGFLLLGFSEHPALERTLFVVVFTSYLLTLVGNTLIILLSVLYPRLHSPMYFFLSDLSFLDLCFTTSCVPQMLVNLWGPKKTISFLGCSVQLFIFLSLGTTECILLTVMAFDRYVAVCQPLHYATIIHPRLCWQLASVAWVMSLVQSIVQTPSTLHLPFCPHQQIDDFLCEVPSLIRLSCGDTSYNEIQLAVSSVIFVVVPLSLILASYGATAQAVLRINSATAWRKAFGTCSSHLTVVTLFYSSVIAVYLQPKNPYAQGRGKFFGLFYAVGTPSLNPLVYTLRNKEIKRALRRLLGKERDSRESWRAA

SEQ ID NO: 2 - protein, 315 aa, Homo sapiens
MVLASGNSSSHPVSFILLGIPGLESFQLWIAFPFCATYAVAVVGNITLLHVIRIDHTLHEPMYLFLAMLAITDLVLSSSTQPKMLAIFWFHAHEIQYHACLIQVFFIHAFSSVESGVLMAMALDCYVATCFPLRHSSILTPSVVIKLGTIVMLRGLLWVSPFCFMVSRMPFCQHQAIPQSYCEHMAVLKLVCADTSISRGYGLFVAFSVAGFDMIVIGMSYVMILRAVLQLPSGEARLKAFSTRASHICVILALYIPALFSFLTYRFGHDVPRVVHILFANLYLLIPPMLNPIIYGVRTKQIGDRVIQGCCGNIP

SEQ ID NO: 3 - protein, 309 aa, Homo sapiens
MGLGNESSLMDFILLGFSDHPRLEAVLFVFVLFFYLLTLVGNFTIIIISYLDPPLHTPMYFFLSNLSLLDICFTTSLAPQTLVNLQRPKKTITYGGCVAQLYISLALGSTECILLADMALDRYIAVCKPLHYVVIMNPRLCQQLASISWLSGLASSLIHATFTLQLPLCGNHRLDHFICEVPALLKLACVDTTVNELVLFVVSVLFVVIPPALISISYGFITQAVLRIKSVEARHKAFSTCSSHLTVVIIFYGTIIYVYLQPSDSYAQDQGKFISLFYTMVTPTLNPIIYTLRNKDMKEALRKLLSGKL

SEQ ID NO: 4 - protein, 321 aa, Homo sapiens
MERKNQTAITEFIILGFSNLNELQFLLFTIFFLTYFCTLGGNILIILTTVTDPHLHTPMYYFLGNLAFIDICYTTSNVPQMMVHLLSKKKSISYVGCVVQLFAFVFFVGSECLLLAAMAYDRYIAICNPLRYSVILSKVLCNQLAASCWAAGFLNSVVHTVLTFCLPFCGNNQINYFFCDIPPLLILSCGNTSVNELALLSTGVFIGWTPFLCIVLSYICIISTILRIQSSEGRRKAFSTCASHLAIVFLFYGSAIFTYVRPISTYSLKKDRLVSVLYSVVTPMLNPIIYTLRNKDIKEAVKTIGSKWQPPISSLDSKLTY

SEQ ID NO: 5 - protein, 49 aa, Homo sapiens
NSVVHTVLTFCLPFCGNNQINYFFCDIPPLLILSCGNTSVNELALLSTG

SEQ ID NO: 6 - protein, 18 aa, Homo sapiens
TVLTFCLPFCGNNQINYF

SEQ ID NO: 7 - DNA, 504 bp, Artificial Sequence
  SYNTHETIC CONSTRUCT
atggactggacctggagcatccttttcttggtggcagcagcaacaggtgcccactcccaggttcagctgctgcagtctggagctgaggtgaagaagcctggggcctcagtgaaggtctcctgcaaggcttctggttacacctttaccagctatggtatcacctgggtgcgacaggcccctggacaagggcttgagtggatgggatggatcagcgcttacaatggtaacacaaactatgcacagaagctccagggcagagtcaccatgaccacagacacatccacgagcacagcctacatggacctgaggagcctgagatctgacgacacggccgtgtattactgtgcgagagcatccgtgccggcccctataactggaactatcatctggttcgacccctggggccaggggacgctggtcaccgtctcctcagggagtgcatccgccccaacccttttccccctcgtctcctgtgagaattccccgtcggatacgagcagcgtg

SEQ ID NO: 8 - protein, 168 aa, Artificial Sequence
  SYNTHETIC CONSTRUCT
MDWTWSILFLVAAATGAHSQVQLLQSGAEVKKPGASVKVSCKASGYTFTSYGITWVRQAPGQGLEWMGWISAYNGNTNYAQKLQGRVTMTTDTSTSTAYMDLRSLRSDDTAVYYCARASVPAPITGTIIWFDPWGQGTLVTVSSGSASAPTLFPLVSCENSPSDTSSV

SEQ ID NO: 9 - protein, 7 aa, Artificial Sequence
  SYNTHETIC CONSTRUCT
GYTFTSY

SEQ ID NO: 10 - protein, 6 aa, Artificial Sequence
  SYNTHETIC CONSTRUCT
SAYNGN

SEQ ID NO: 11 - protein, 16 aa, Artificial Sequence
  SYNTHETIC CONSTRUCT
ASVPAPITGTIIWFDP

SEQ ID NO: 12 - DNA, 593 bp, Artificial Sequence
  SYNTHETIC CONSTRUCT
atggcctggaccgttctcctcctcggcctcctctctcactgcacaggctctgtgacctcctacgtgctgactcagccaccctcggtgtcagtggccccaggacagacggccaggattacctgtgggggaaacaacattggaagtaaaagtgtacactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagcta

SEQ ID NO: 13 - protein, 139 aa, Artificial Sequence
  SYNTHETIC CONSTRUCT
MAWTVLLLGLLSHCTGSVTSYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGQPKAAPSVTLF

SEQ ID NO: 14 - protein, 11 aa, Artificial Sequence
  SYNTHETIC CONSTRUCT
GGNNIGSKSVH

SEQ ID NO: 15 - protein, 7 aa, Artificial Sequence
  SYNTHETIC CONSTRUCT
DDSDRPS

SEQ ID NO: 16 - protein, 11 aa, Artificial Sequence
  SYNTHETIC CONSTRUCT
QVWDSSSDHVV

SEQ ID NO: 17 - protein, 496 aa, Artificial Sequence
  SYNTHETIC CONSTRUCT
MDWTWSILFLVAAATGAHSQVQLLQSGAEVKKPGASVKVSCKASGYTFTSYGITWVRQAPGQGLEWMGWISAYNGNTNYAQKLQGRVTMTTDTSTSTAYMDLRSLRSDDTAVYYCARASVPAPITGTIIWFDPWGQGTLVTVSSGGGGSGGGGSGGGGSGSVTSYVLTQPPSVSVAPGQTARITCGGNNIGSKSVHWYQQKPGQAPVLVVYDDSDRPSGIPERFSGSNSGNTATLTISRVEVVFGGGTKLTVLKVENHTACHCSTCYYHKSASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - DNA, 1491 bp, Artificial Sequence
  SYNTHETIC CONSTRUCT
atggactggacctggagcatccttttcttggtggcagcagcaacaggtgcccactcccaggttcagctgctgcagtctggagctgaggtgaagaagcctggggcctcagtgaaggtctcctgcaaggcttctggttacacctttaccagctatggtatcacctgggtgcgacaggcccctggacaagggcttgagtggatgggatggatcagcgcttacaatggtaacacaaactatgcacagaagctccagggcagagtcaccatgaccacagacacatccacgagcacagcctacatggacctgaggagcctgagatctgacgacacggccgtgtattactgtgcgagagcatccgtgccggcccctataactggaactatcatctggttcgacccctggggccaggggacgctggtcaccgtctcctcaggtggaggcggttcaggcggcggtggctctggcggtggcggatcgggctctgtgacctcctacgtgctgactcagccaccctcggtgtcagtggccccaggacagacggccaggattacctgtgggggaaacaacattggaagtaaaagtgtacactggtaccagcagaagccaggccaggcccctgtgctggtcgtctatgatgatagcgaccggccctcagggatccctgagcgattctctggctccaactctgggaacacggccaccctgaccatcagcagggtcgaagtggtattcggcggagggaccaagctgaccgtcctaaaggtggaaaaccacaccgcctgccactgcagcacatgctactaccacaagagcgctagcaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 19 - DNA, 618 bp, Artificial Sequence
  SYNTHETIC CONSTRUCT
atggaactggggctccgctgggttttccttgttgctattttagaaggtgtccagtgtgaggtgcagctggtggagtctgggggaggcctggtcaagcctggggggtccctgagactctcctgtgcagcctctggattcaccttcagtagctatagcatgacctgggtccgccaggctccagggaaggggctggactgggtctcatccattagtagtagtagtagttacatatactacgcagactcattgaagggccgattcaccatctccagagacaacgccaagaactcactgtatctacaaattaacagcctgagagtcgaggacacggctgtgtattactgtgcgagagatccttcattatggccgaaaccccactactactacgacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcagga

SEQ ID NO: 20 - protein, 145 aa, Artificial Sequence
  SYNTHETIC CONSTRUCT
MELGLRWVFLVAILEGVQCEVQLVESGGGLVKPGGSLRLSCAASGFTFSSYSMTWVRQAPGKGLDWVSSISSSSSYIYYADSLKGRFTISRDNAKNSLYLQINSLRVEDTAVYYCARDPSLWPKPHYYYDGMDVWGQGTTVTVSS

SEQ ID NO: 21 - protein, 7 aa, Artificial Sequence
  SYNTHETIC CONSTRUCT
GFTFSSY

SEQ ID NO: 22 - protein, 6 aa, Artificial Sequence
  SYNTHETIC CONSTRUCT
SSSSSY

SEQ ID NO: 23 - protein, 15 aa, Artificial Sequence
  SYNTHETIC CONSTRUCT
DPSLWPKPHYYYDGM

SEQ ID NO: 24 - DNA, 521 bp, Artificial Sequence
  SYNTHETIC CONSTRUCT
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgttagccgcagctatttagcctggtaccagcagaaacctggccaggctcccaggctcctcatctatggtgcatccagcagggccactggcatcccagacaggttcagtggcagtgagtctgggacagacttcactctcaccatcaacagactggagcctgaagattttgcactgtattactgtcagctatatggtaactctctgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgc

SEQ ID NO: 25 - protein, 128 aa, Artificial Sequence
  SYNTHETIC CONSTRUCT
METPAQLLFLLLLWLPDTTGEIVLTQSPGTLSLSPGERATLSCRASQSVSRSYLAWYQQKPGQAPRLLIYGASSRATGIPDRFSGSESGTDFTLTINRLEPEDFALYYCQLYGNSLITFGQGTRLEIK

SEQ ID NO: 26 - protein, 12 aa, Artificial Sequence
  SYNTHETIC CONSTRUCT
RASQSVSRSYLA

SEQ ID NO: 27 - protein, 7 aa, Artificial Sequence
  SYNTHETIC CONSTRUCT
GASSRAT

SEQ ID NO: 28 - protein, 9 aa, Artificial Sequence
  SYNTHETIC CONSTRUCT
QLYGNSLIT

SEQ ID NO: 29 - DNA, 1473 bp, Artificial Sequence
  SYNTHETIC CONSTRUCT
atggactggacctggaggatcctcttcttggtggcagcagccacaggagcccacatggcccaggtacagctgcagcagtcaggagcagaggtgaaaaagccgggggaatctctgaagatctcctgtaagggttctggatacatctttaccggctactatatgcactgggtgcgacaggcccctggacaacggcctgagtggctgggacggatgaacgcaaaaagtggtgaggcagactctgcacagagatttcagggcagggtcaccatgaccagagacacgtccatcaatacagcctacatggaactgagagacctgagatctgacgacacggccgtgtattattgtacgagaggtcccctgctctggggccagggaaccctggtcaccgtctcgagtggtggtggcggttctggtggtggtggtagcggtggcggtggtagtggcggtggcggtgctagccagcctgtgctgactcagccaccctcagcgtctgggacccccgggcagagggtcaccatctcctgttctggaagcagctccaacatcgggagtaattttgtatcctggtaccagcagctcccaggaacggcccccaaactcctcatctataggaataatcagcggccctcaggggtccctgaccgattctctggctccaagactggcacctcagcctccctggccatcagtgggctccggtccgaggatgaggctgattattactgtgcagcatgggatgacagtgtgagaggtccggtgttcggcggagggaccgagctgaccgtcctagcggccgcaaccaccacccctgcccctagacctccaacacccgcccctacaatcgcctcccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaagaggaagaggggggctgcgaactgagagtgaagttcagcagaagcgccgacgcccctgcctacaagcagggccagaaccagctgtacaacgagctgaacctgggcagacgggaagagtacgacgtgctggacaagcggagaggcagggaccctgagatgggcggaaagcccagacggaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccccagataa

SEQ ID NO: 30 - protein, 490 aa, Artificial Sequence
  SYNTHETIC CONSTRUCT
MDWTWRILFLVAAATGAHMAQVQLQQSGAEVKKPGESLKISCKGSGYIFTGYYMHWVRQAPGQRPEWLGRMNAKSGEADSAQRFQGRVTMTRDTSINTAYMELRDLRSDDTAVYYCTRGPLLWGQGTLVTVSSGGGGSGGGGSGGGGSGGGGASQPVLTQPPSASGTPGQRVTISCSGSSSNIGSNFVSWYQQLPGTAPKLLIYRNNQRPSGVPDRFSGSKTGTSASLAISGLRSEDEADYYCAAWDDSVRGPVFGGGTELTVLAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 8 aa, Artificial Sequence
  SYNTHETIC CONSTRUCT
GYIFTGYY

SEQ ID NO: 32 - protein, 6 aa, Artificial Sequence
  SYNTHETIC CONSTRUCT
NAKSGE

SEQ ID NO: 33 - protein, 4 aa, Artificial Sequence
  SYNTHETIC CONSTRUCT
GPLL

SEQ ID NO: 34 - protein, 113 aa, Artificial Sequence
  SYNTHETIC CONSTRUCT
QVQLQQSGAEVKKPGESLKISCKGSGYIFTGYYMHWVRQAPGQRPEWLGRMNAKSGEADSAQRFQGRVTMTRDTSINTAYMELRDLRSDDTAVYYCTRGPLLWGQGTLVTVSS

SEQ ID NO: 35 - protein, 13 aa, Artificial Sequence
  SYNTHETIC CONSTRUCT
SSGSSNIGSNFVS

SEQ ID NO: 36 - protein, 7 aa, Artificial Sequence
  SYNTHETIC CONSTRUCT
RNNQRPS

SEQ ID NO: 37 - protein, 11 aa, Artificial Sequence
  SYNTHETIC CONSTRUCT
AAWDDSVRGPV

SEQ ID NO: 38 - protein, 113 aa, Artificial Sequence
  SYNTHETIC CONSTRUCT
QPVLTQPPSASGTPGQRVTISCSGSSSNIGSNFVSWYQQLPGTAPKLLIYRNNQRPSGVPDRFSGSKTGTSASLAISGLRSEDEADYYCAAWDDSVRGPVFGGGTELTVLAAA

SEQ ID NO: 39 - protein, 249 aa, Artificial Sequence
  SYNTHETIC CONSTRUCT
MAQVQLQQSGAEVKKPGESLKISCKGSGYIFTGYYMHWVRQAPGQRPEWLGRMNAKSGEADSAQRFQGRVTMTRDTSINTAYMELRDLRSDDTAVYYCTRGPLLWGQGTLVTVSSGGGGSGGGGSGGGGSGGGGASQPVLTQPPSASGTPGQRVTISCSGSSSNIGSNFVSWYQQLPGTAPKLLIYRNNQRPSGVPDRFSGSKTGTSASLAISGLRSEDEADYYCAAWDDSVRGPVFGGGTELTVLAAA

SEQ ID NO: 40 - protein, 115 aa, Artificial Sequence
  SYNTHETIC CONSTRUCT
MAQVQLQQSGAEVKKPGESLKISCKGSGYIFTGYYMHWVRQAPGQRPEWLGRMNAKSGEADSAQRFQGRVTMTRDTSINTAYMELRDLRSDDTAVYYCTRGPLLWGQGTLVTVSS

SEQ ID NO: 41 - protein, 8 aa, Artificial Sequence
  SYNTHETIC CONSTRUCT
GYIFTGYY

SEQ ID NO: 42 - protein, 8 aa, Artificial Sequence
  SYNTHETIC CONSTRUCT
MNAKSGEA

SEQ ID NO: 43 - protein, 6 aa, Artificial Sequence
  SYNTHETIC CONSTRUCT
TRGPLL

SEQ ID NO: 44 - protein, 110 aa, Artificial Sequence
  SYNTHETIC CONSTRUCT
QPVLTQPPSASGTPGQRVTISCSGSSSNIGSNFVSWYQQLPGTAPKLLIYRNNQRPSGVPDRFSGSKTGTSASLAISGLRSEDEADYYCAAWDDSVRGPVFGGGTELTVL

SEQ ID NO: 45 - protein, 8 aa, Artificial Sequence
  SYNTHETIC CONSTRUCT
SSNIGSNF

SEQ ID NO: 46 - protein, 3 aa, Artificial Sequence
  SYNTHETIC CONSTRUCT
RNN

SEQ ID NO: 47 - protein, 11 aa, Artificial Sequence
  SYNTHETIC CONSTRUCT
AAWDDSVRGPV

SEQ ID NO: 48 - protein, 122 aa, Artificial Sequence
  SYNTHETIC CONSTRUCT
EVQLVQSGAEVKKPGASVKVSCKASGYTFTSNYMHWVRQAPGQGLEWMGIINPSGGRTSYAQKFQGRVTMTRDTSTGTVYMELSSLRSEDTAVYYCARSHCSGGSCYSIDYWGQGTLVTVSS

SEQ ID NO: 49 - protein, 25 aa, Artificial Sequence
  SYNTHETIC CONSTRUCT
EVQLVQSGAEVKKPGASVKVSCKAS

SEQ ID NO: 50 - protein, 17 aa, Artificial Sequence
  SYNTHETIC CONSTRUCT
MHWVRQAPGQGLEWMGI

SEQ ID NO: 51 - protein, 38 aa, Artificial Sequence
  SYNTHETIC CONSTRUCT
SYAQKFQGRVTMTRDTSTGTVYMELSSLRSEDTAVYYC

SEQ ID NO: 52 - protein, 11 aa, Artificial Sequence
  SYNTHETIC CONSTRUCT
WGQGTLVTVSS

SEQ ID NO: 53 - protein, 8 aa, Artificial Sequence
  SYNTHETIC CONSTRUCT
GYTFTSNY

SEQ ID NO: 54 - protein, 8 aa, Artificial Sequence
  SYNTHETIC CONSTRUCT
INPSGGRT

SEQ ID NO: 55 - protein, 15 aa, Artificial Sequence
  SYNTHETIC CONSTRUCT
ARSHCSGGSCYSIDY

SEQ ID NO: 56 - protein, 111 aa, Artificial Sequence
  SYNTHETIC CONSTRUCT
QPVLTQSSSASASLGSSVKLTCTLSSGHSGYIIAWHQQQPGKAPRYLMKVEGSGSYNKGSGIPERFSGSSSGADRYLTISNLQSEDEADYYCETWDSNTHVFGTGTKVTVL

SEQ ID NO: 57 - protein, 25 aa, Artificial Sequence
  SYNTHETIC CONSTRUCT
QPVLTQSSSASASLGSSVKLTCTLS

SEQ ID NO: 58 - protein, 17 aa, Artificial Sequence
  SYNTHETIC CONSTRUCT
IAWHQQQPGKAPRYLMK

SEQ ID NO: 59 - protein, 36 aa, Artificial Sequence
  SYNTHETIC CONSTRUCT
NKGSGIPERFSGSSSGADRYLTISNLQSEDEADYYC

SEQ ID NO: 60 - protein, 10 aa, Artificial Sequence
  SYNTHETIC CONSTRUCT
FGTGTKVTVL

SEQ ID NO: 61 - protein, 7 aa, Artificial Sequence
  SYNTHETIC CONSTRUCT
SGHSGYI

SEQ ID NO: 62 - protein, 7 aa, Artificial Sequence
  SYNTHETIC CONSTRUCT
VEGSGSY

SEQ ID NO: 63 - protein, 9 aa, Artificial Sequence
  SYNTHETIC CONSTRUCT
ETWDSNTHV

SEQ ID NO: 64 - protein, 498 aa, Artificial Sequence
  SYNTHETIC CONSTRUCT
MDWTWRVFCLLAVAPGAHSEVQLVQSGAEVKKPGASVKVSCKASGYTFTSNYMHWVRQAPGQGLEWMGIINPSGGRTSYAQKFQGRVTMTRDTSTSTVYMELSSLRSEDTAVYYCARSHCSGGSCYSIDYWGQGTLVTVSSGGGGSGGGGSGGGGSGGGGQPVLTQSSSASASLGSSVKLTCTLSSGHSGYIIAWHQQQPGKAPRYLMKVEGSGSYNKGSGVPDRFSGSSSGADRYLTISNLQFEDEADYYCETWDSNTHVYFGTGTKVTVLAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 123 aa, Artificial Sequence
  SYNTHETIC CONSTRUCT
SEVQLVQSGAEVKKPGASVKVSCKASGYTFTSNYMHWVRQAPGQGLEWMGIINPSGGRTSYAQKFQGRVTMTRDTSTSTVYMELSSLRSEDTAVYYCARSHCSGGSCYSIDYWGQGTLVTVSS

SEQ ID NO: 66 - protein, 115 aa, Artificial Sequence
  SYNTHETIC CONSTRUCT
QPVLTQSSSASASLGSSVKLTCTLSSGHSGYIIAWHQQQPGKAPRYLMKVEGSGSYNKGSGVPDRFSGSSSGADRYLTISNLQFEDEADYYCETWDSNTHVYFGTGTKVTVLAAA